(19) United States
(12) Patent Application Publication
(10) Pub. No.: US 2023/0322700 A1
(43) Pub. Date: Oct. 12, 2023

(54) TETRASTYRENE-BASED COMPOUND AND APPLICATION THEREOF AND ELECTRONIC DEVICE USING THE SAME

(71) Applicant: Wuhan China Star Optoelectronics Semiconductor Display Technology Co., Ltd., Wuhan, Hubei (CN)

(72) Inventors: Keyan BAI, Wuhan, Hubei (CN); Jiajia LUO, Wuhan, Hubei (CN)

(73) Assignee: Wuhan China Star Optoelectronics Semiconductor Display Technology Co., Ltd., Wuhan, Hubei (CN)

(21) Appl. No.: 17/263,923

(22) PCT Filed: Nov. 30, 2020
(86) PCT No.: PCT/CN2020/132600

§ 371 Date: Jun. 28, 2023

(30) Foreign Application Priority Data
Nov. 11, 2020 (CN) 202011252257.8

(51) Int. Cl. C07D 307/91; C07D 209/86; C07D 407/12; C07D 311/88; C07D 405/10; C07D 219/10; H10K 50/11; H10K 50/15; H10K 50/17; H10K 85/60; C09K 11/06

(52) U.S. Cl. CPC C07D 307/91; C07D 209/86; C07D 407/12; C07D 311/88; C07D 405/10; C07D 219/10; C09K 2211/1018; H10K 50/15; H10K 50/171; H10K 85/6572; H10K 85/6574; C09K 11/06; H10K 50/11

(57) ABSTRACT

The present application discloses a tetrastyrene-based compound, an application thereof, and an electronic device using the same. The tetrastyrene-based compound has a general structural formula as shown in the following Formula 1:

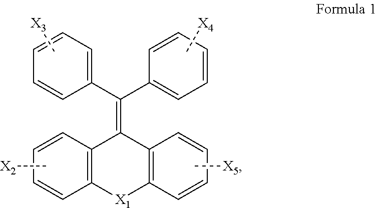

The tetrastyrene-based compound includes an aromatic amine and a rigid tetrastyrene structure, wherein the aromatic amine can effectively improve the hole injection and transport performance, and the rigid tetrastyrene structure is conducive to the formation of evaporation materials of melting type.

### FIELD OF INVENTION

The present application relates to the technical field of organic optoelectronic materials. More specifically, the present application relates to a tetrastyrene-based compound and an application thereof and electronic devices using the same.

### DESCRIPTION OF PRIOR ART

Organic electronic devices refer to devices composed of an anode, a cathode, and an organic layer sandwiched between the anode and the cathode, and including organic light-emitting diodes, organic solar cells, organic semiconductors, organic crystals, etc. Working principle of the organic electronic devices, for example, the organic light-emitting diodes, is to apply an external voltage on an electrode to inject holes and electrons into the organic layer to form excitons, thereby radiating light; or working principle of the organic electronic devices, for example, the organic solar cells, is that the external light source is absorbed by organic materials to form excitons, and the excitons are separated into holes and electrons which are transferred to electrodes and stored. The following description mainly focuses on the organic light-emitting diodes.

Organic light-emitting diodes are devices that convert electrical energy into light energy, and their structure usually includes an anode, a cathode, and one or more layers of organic materials interposed therebetween. The organic material layers are divided into a hole injection material layer, a hole transport material layer, an electron injection material layer, an electron transport material layer, and a light-emitting material layer according to functions. In addition, light-emitting materials are divided into blue, sky blue, green, yellow, red, and deep red light-emitting materials according to their luminous colors.

Evaluation indicators of the organic light-emitting diodes are mainly voltage, efficiency, and service life, and how to develop low-voltage, high-efficiency and long-life organic light-emitting diode devices has always been the goal pursued by the R&D and business circles, which requires high mobility of electrons/holes injection and transport materials, and also require high-efficiency light-emitting materials and an effective balance of electrons and holes in the device. In addition, from the perspective of the mass production of organic materials, the vapor deposition type (sublimation type or melting type), decomposition temperature, glass transition temperature, and outgassing of the material must also be considered. Especially in mass production, thicker hole transport materials need to be deposited, wherein materials of sublimation type will seriously impact the uniformity of the film thickness in mass production. Therefore, the development of hole transport materials of melting type has become an important direction.

### SUMMARY OF INVENTION

Embodiments of the present application innovatively provide a tetrastyrene-based compound and an application thereof and an electronic device using the same. The organic material includes an aromatic amine and a rigid tetrastyrene structure, wherein the aromatic amine can effectively improve the hole injection and transport performance, and the rigid tetrastyrene structure is conducive to the formation of evaporation materials of melting type.

In order to achieve the above technical objectives, on the one hand, an embodiment of the present application discloses a tetrastyrene-based compound, having a general structural formula as shown in the following Formula 1:

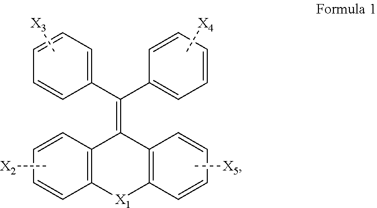

wherein, in Formula 1, X1 is selected from,

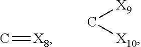

### O, N—H₁₁, or S;

X2, X3, X4, and X5 are each independently selected from an arylamine with a general structural formula as shown in Formula 2, a hydrogen, an alkyl group having 1 to 22 carbon atoms, an alkoxy group having 1 to 22 carbon atoms, or a heteroalkyl group having 1 to 22 carbon atoms, a mono- or multi-substituted or unsubstituted aryl group, or a substituted or unsubstituted heteroaryl group, wherein a heteroatom of the heteroalkyl group is O, N, F, S, or Si, a heteroatom of the heteroaryl group is Si, Ge, N, P, O, S, or Se, and at least one of X2, X3, X4, and X5 is an arylamine with the general structural formula as shown in Formula 2:

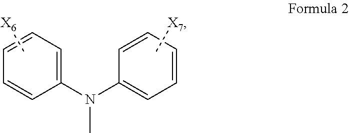

X6, X7, X8, X9, X10, and X11 are each independently selected from hydrogen, an alkyl group, an alkoxy group having 1 to 22 carbon atoms, or a heteroalkyl group having 1 to 22 carbon atoms, a mono- or multi-substituted or unsubstituted aryl group, or a substituted or unsubstituted heteroaryl group, wherein a heteroatom of the heteroalkyl group is O, N, F, S, or Si, and a heteroatom of the heteroaryl group is Si, Ge, N, P, O, S, or Se, or at least two of X2, X3, X4, X5, X6, X7, X8, X9, X10, and X11 are connected to each other to form a monocyclic ring or fused ring of an aromatic ring or a heterocyclic ring when the at least two of X2, X3, X4, X5, X6, X7, X8, X9, X10, and X11 are adjacent aryl groups or heteroaryl groups; and
when expressed as “substituted or unsubstituted”, optional substituents of each of the aryl group and the heteroaryl group are each independently selected from H, halogen, —OH, —SH, —CN, —NO₂, and an alkylthio group having 1 to 15 carbon atoms, an alkyl group having 1 to 40 carbon atoms, or a substituted alkyl group having 1 to 40 carbon atoms.

Further, as for the tetrastyrene-based compound, a general structural formula of X1 is

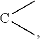

and a general structural formula of the tetrastyrene-based compound is shown in the following Formula 3:

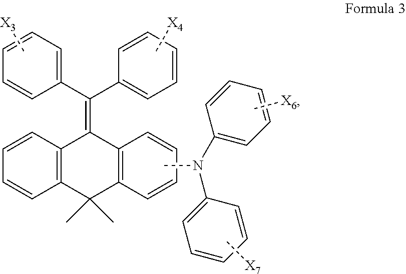

wherein, in Formula 3, X3, X4, X6, and X7 are each independently selected from a mono- or multi-substituted, or unsubstituted aryl group, or a substituted or unsubstituted heteroaryl group, or at least two of X3, X4, X6, and X7 are connected to each other to form a monocyclic ring or fused ring of an aromatic ring or a heterocyclic ring when the at least two of X3, X4, X6, and X7 are adjacent aryl groups or heteroaryl groups.

Further, as for the tetrastyrene-based compound, a structure of the tetrastyrene-based compound is represented by any one of the following general Formulas 301-320:

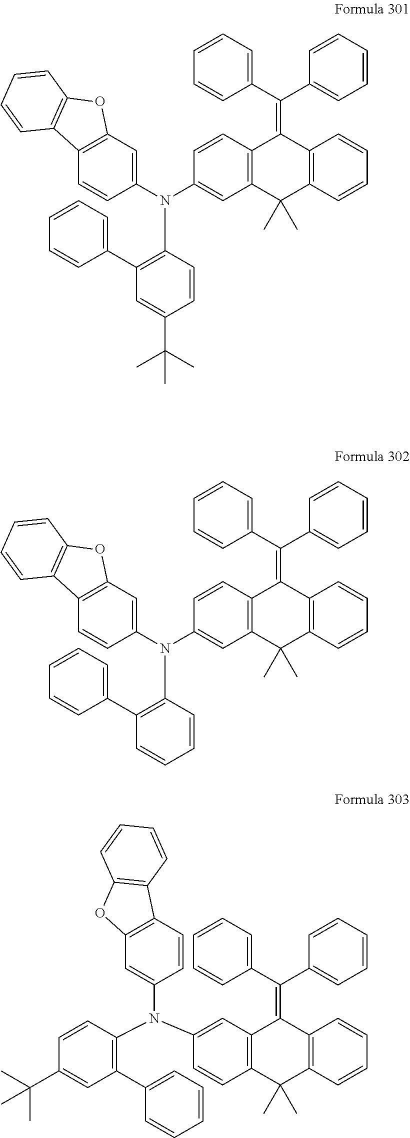

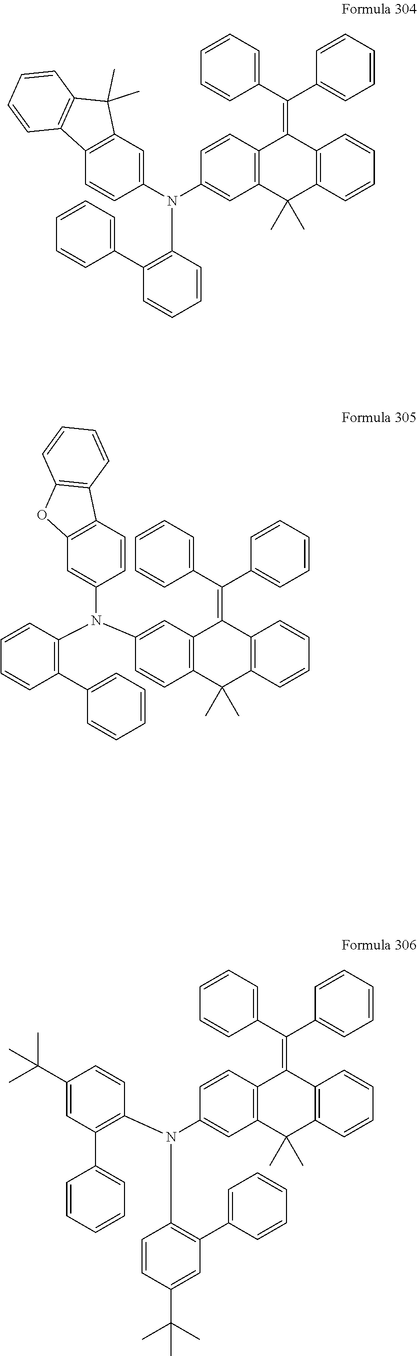

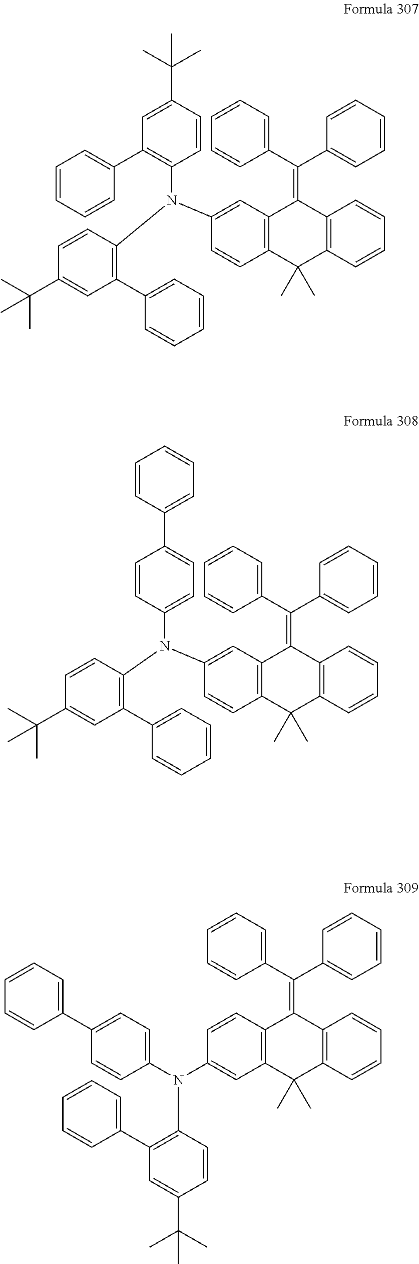

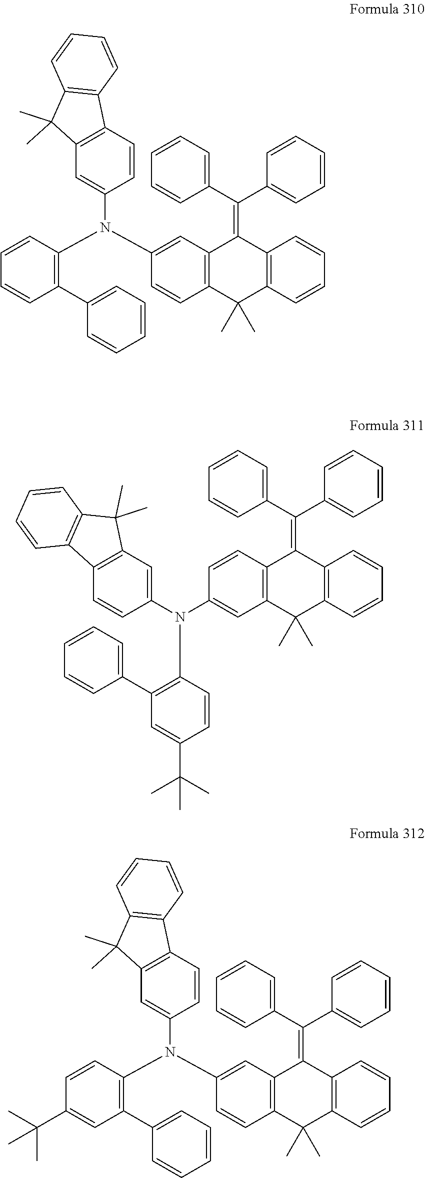

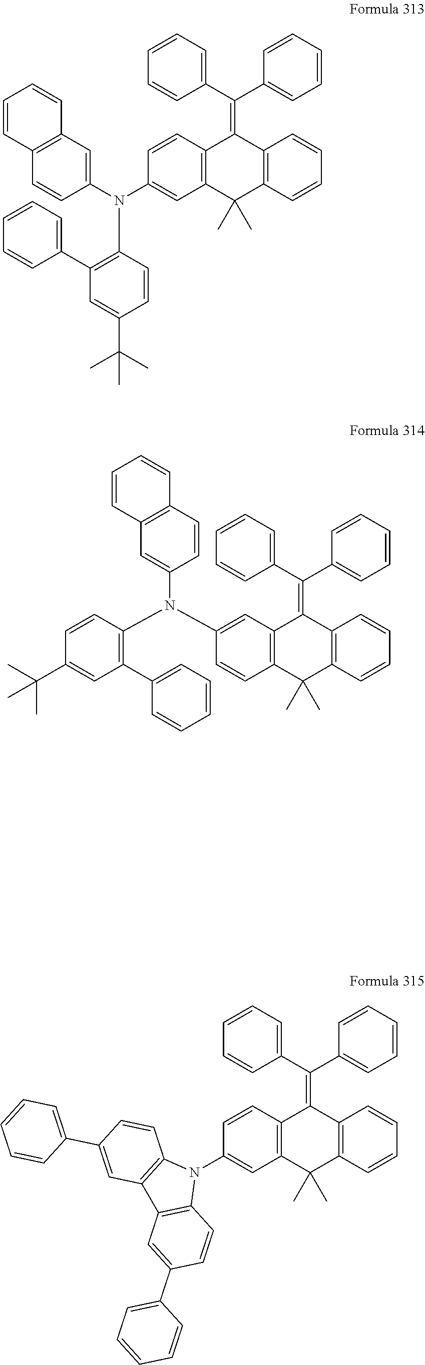

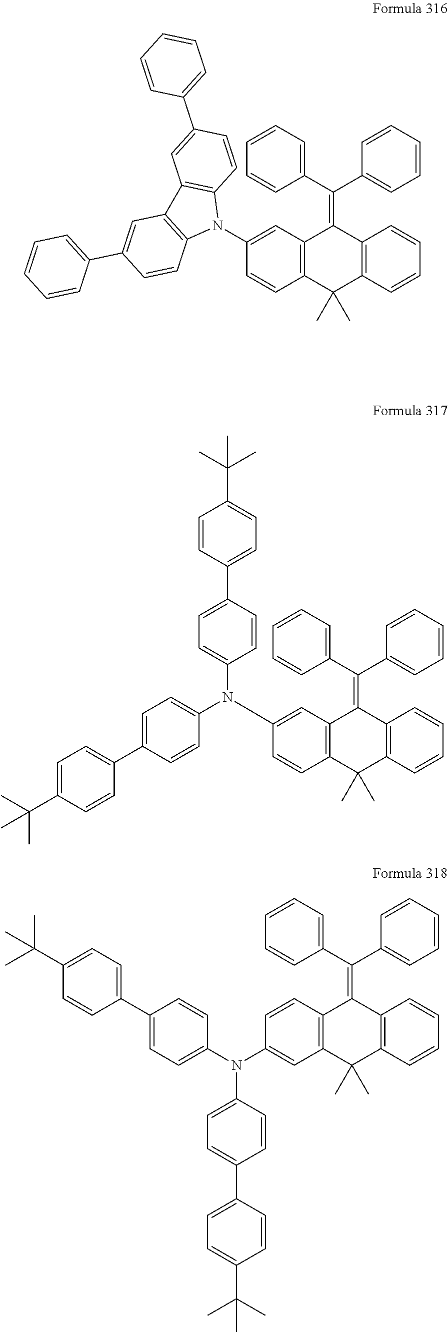

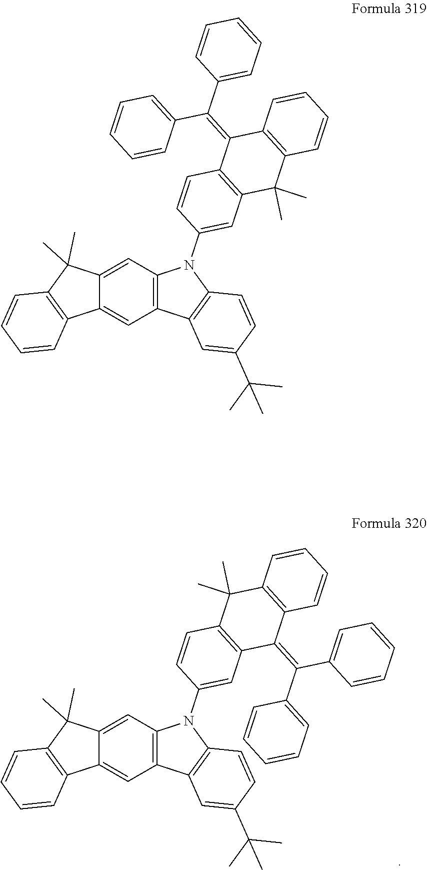

Further, as for the tetrastyrene-based compound, a general structural formula of X1 is O, and a general structural formula of the tetrastyrene-based compound is shown in the following Formula 4:

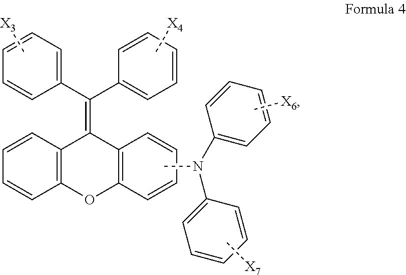

wherein, in Formula, X3, X4, X6, and X7 are each independently selected from a mono- or multi-substituted, or unsubstituted aryl group, or a substituted or unsubstituted heteroaryl group, or at least two of X3, X4, X6, and X7 are connected to each other to form a monocyclic ring or fused ring of an aromatic ring or a heterocyclic ring when the at least two of X3, X4, X6, and X7 are adjacent aryl groups or heteroaryl groups.

Further, as for the tetrastyrene-based compound, a structure of the tetrastyrene-based compound is represented by any one of the following general Formulas 401-420:

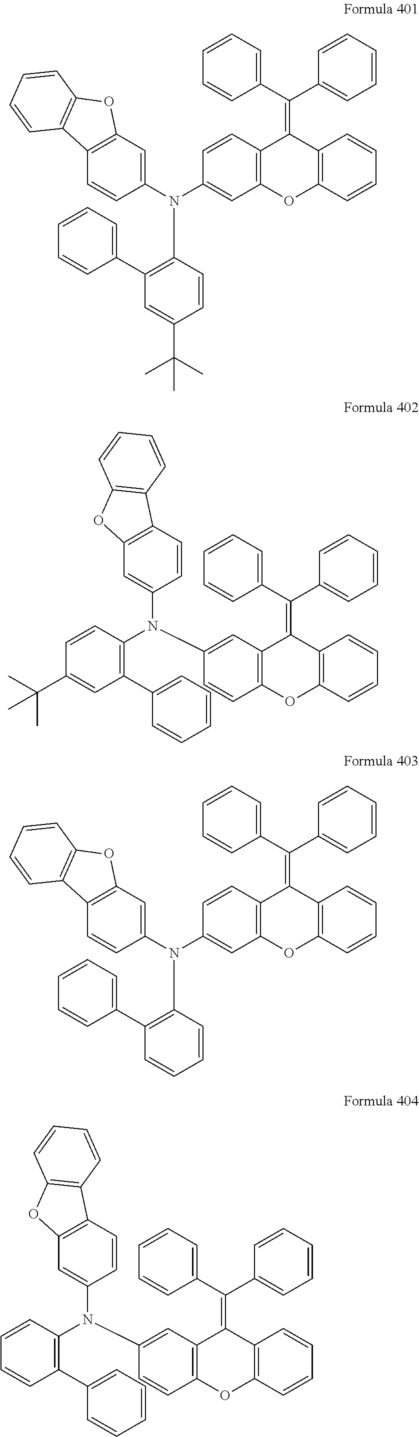

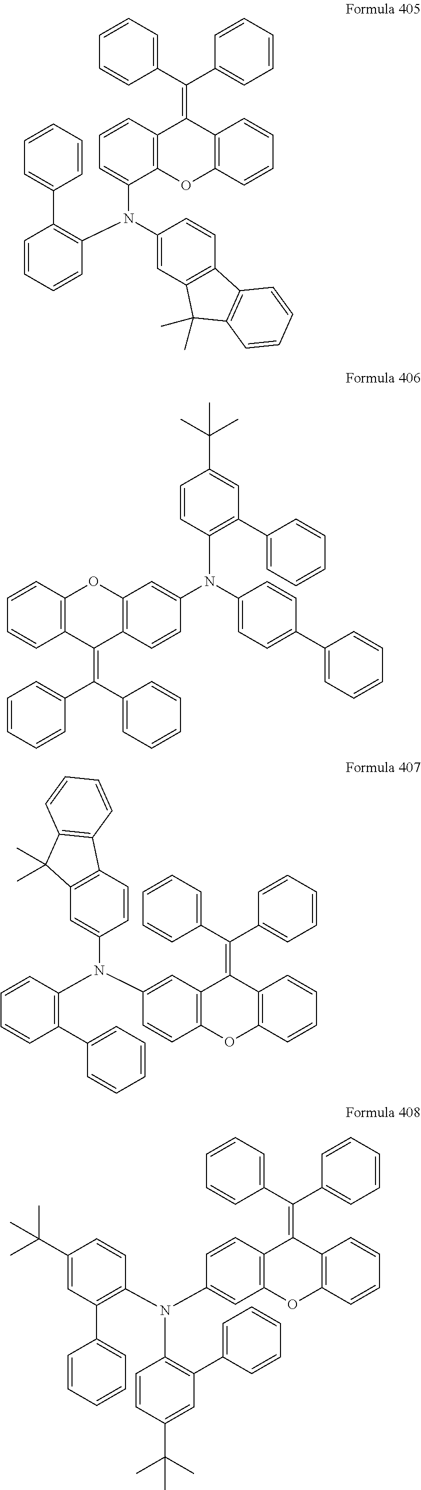

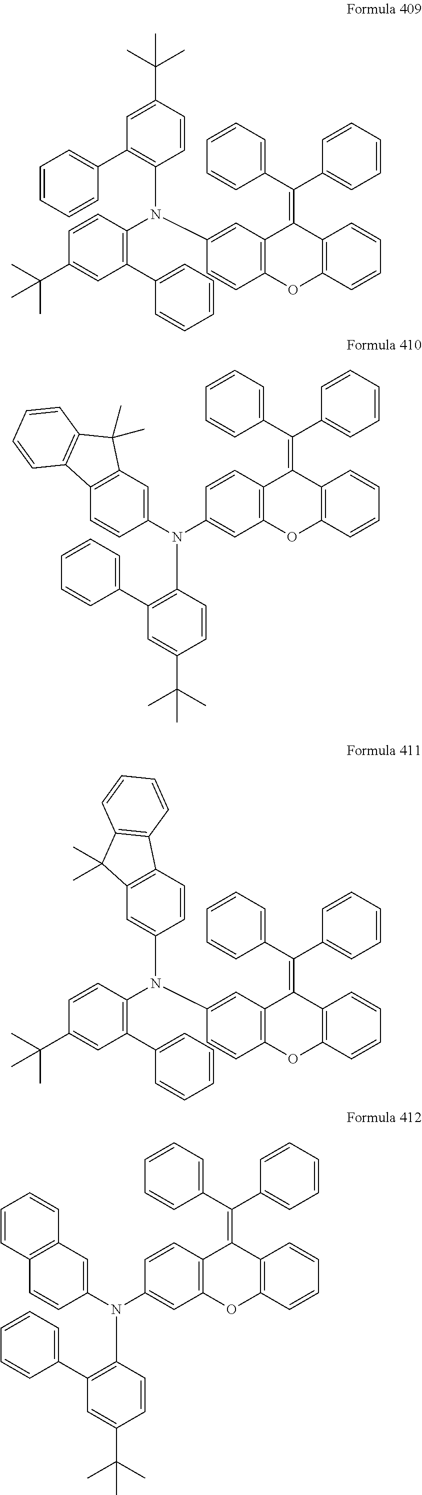

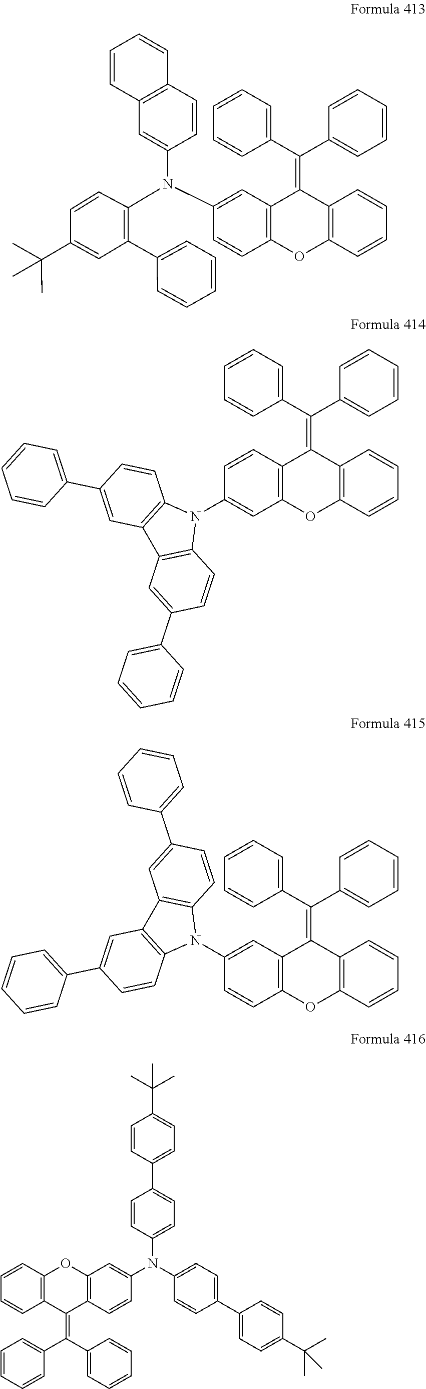

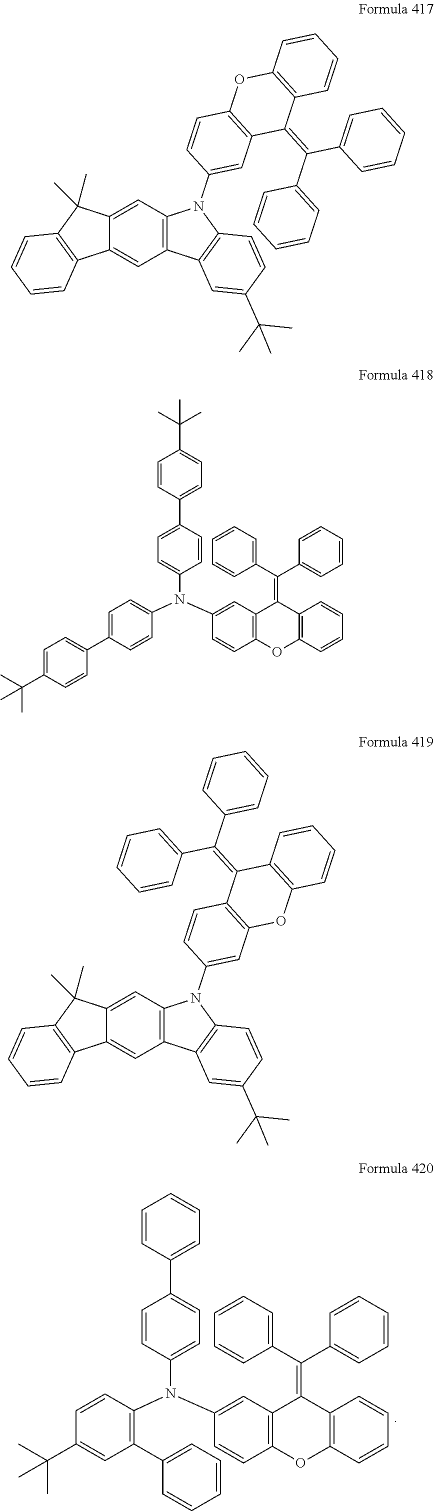

Further, as for the tetrastyrene-based compound, a general structural formula of X1 is N-ph, and a general structural formula of the tetrastyrene-based compound is shown in Formula 5 below:

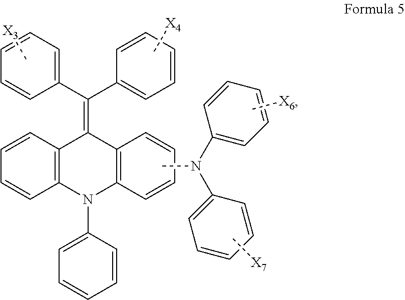

wherein, in Formula 5, X3, X4, X6, and X7 are each independently selected from a mono- or multi-substituted, or unsubstituted aryl group, or a substituted or unsubstituted heteroaryl group, or at least two of X3, X4, X6, and X7 are connected to each other to form a monocyclic ring or fused ring of an aromatic ring or a heterocyclic ring when the at least two of X3, X4, X6, and X7 are adjacent aryl groups or heteroaryl groups.

Further, as for the tetrastyrene-based compound, a structure of the tetrastyrene-based compound is represented by any one of the following general Formulas 501-520:

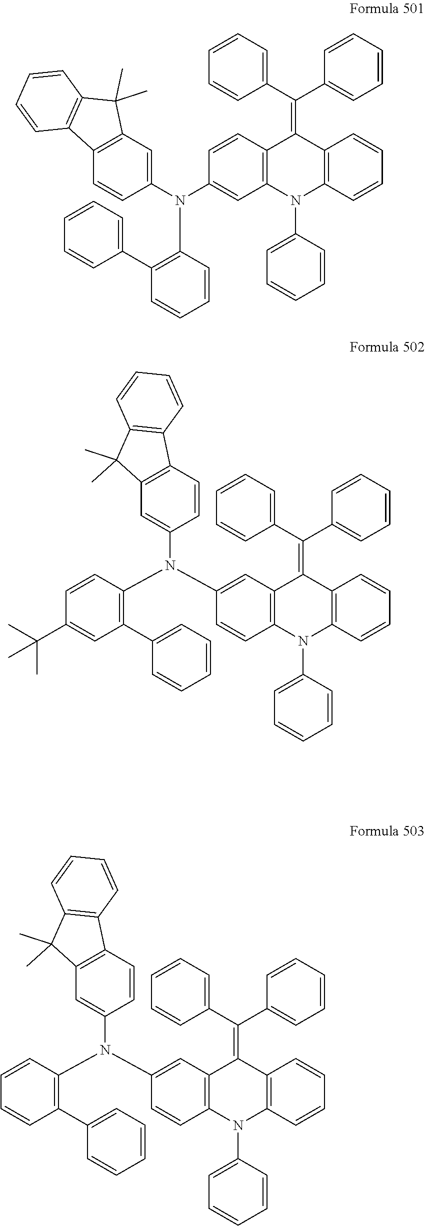

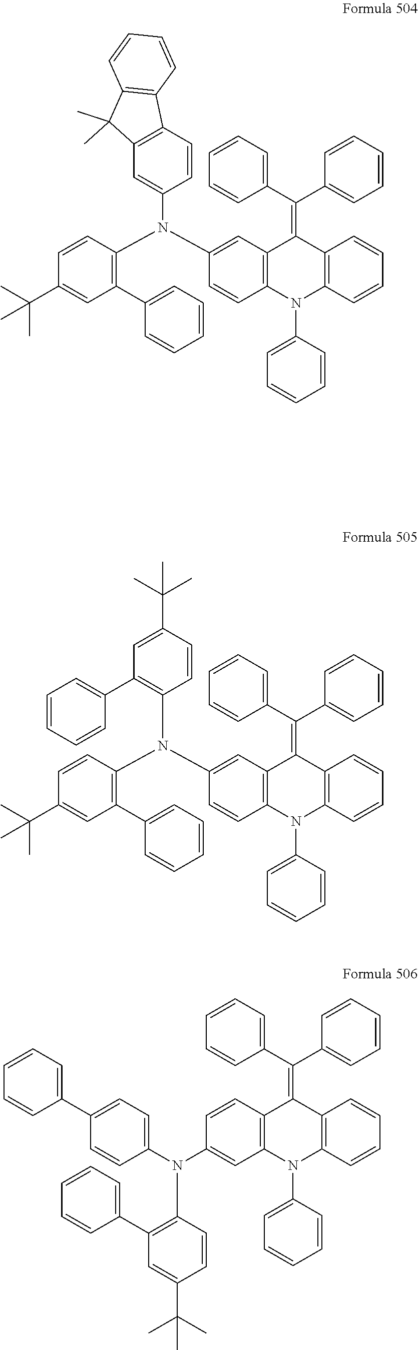

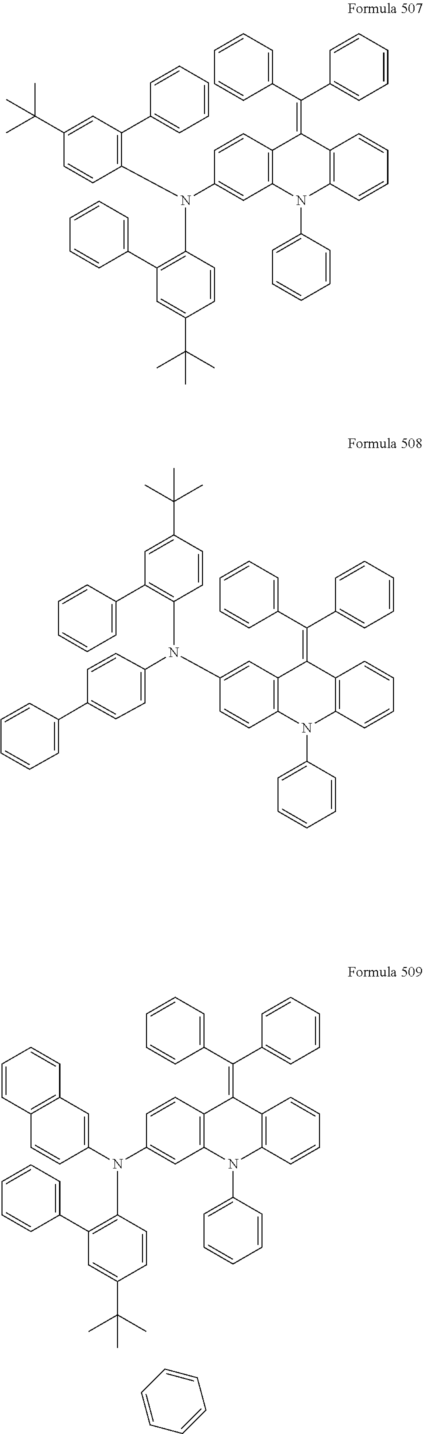

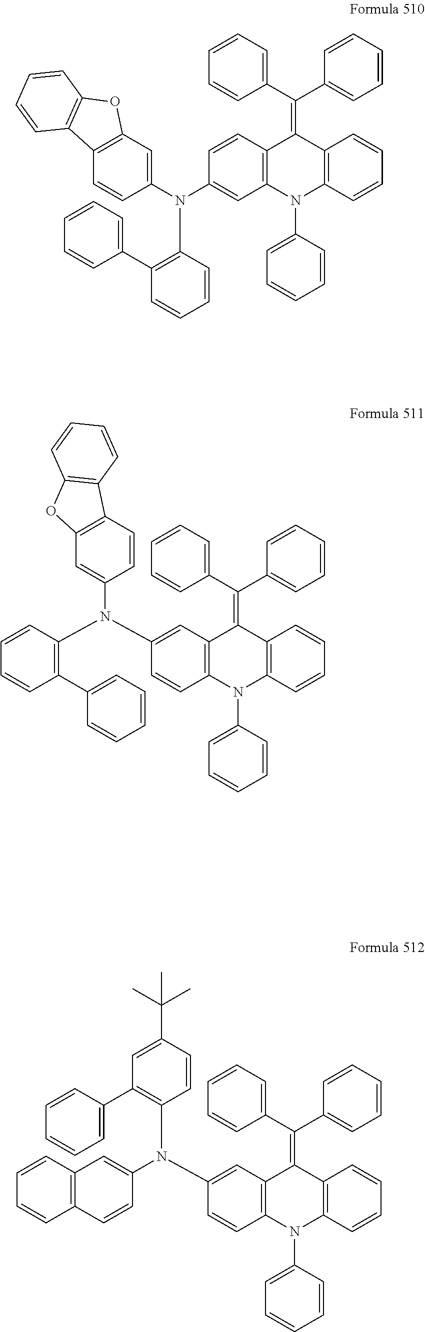

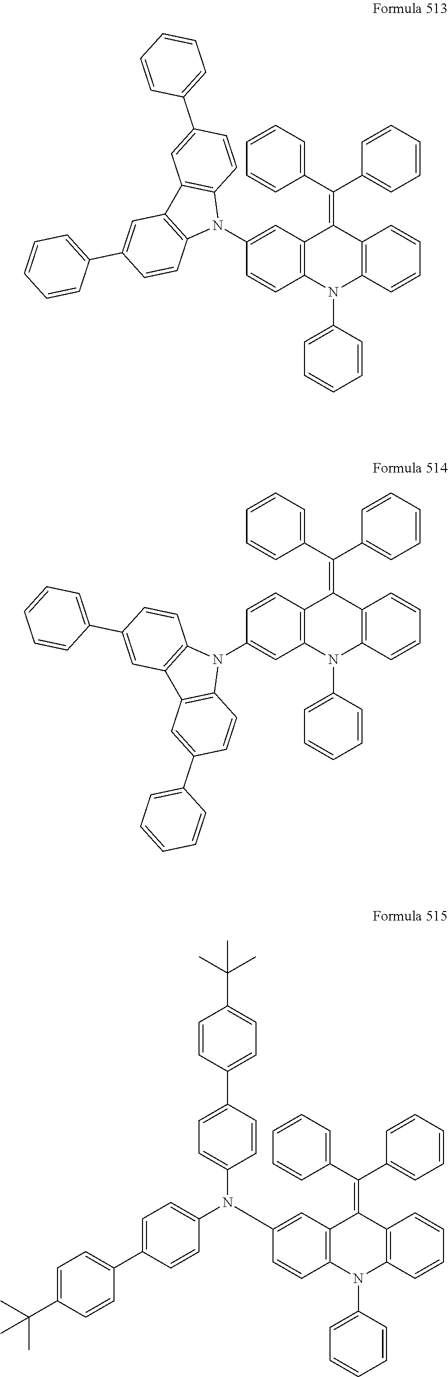

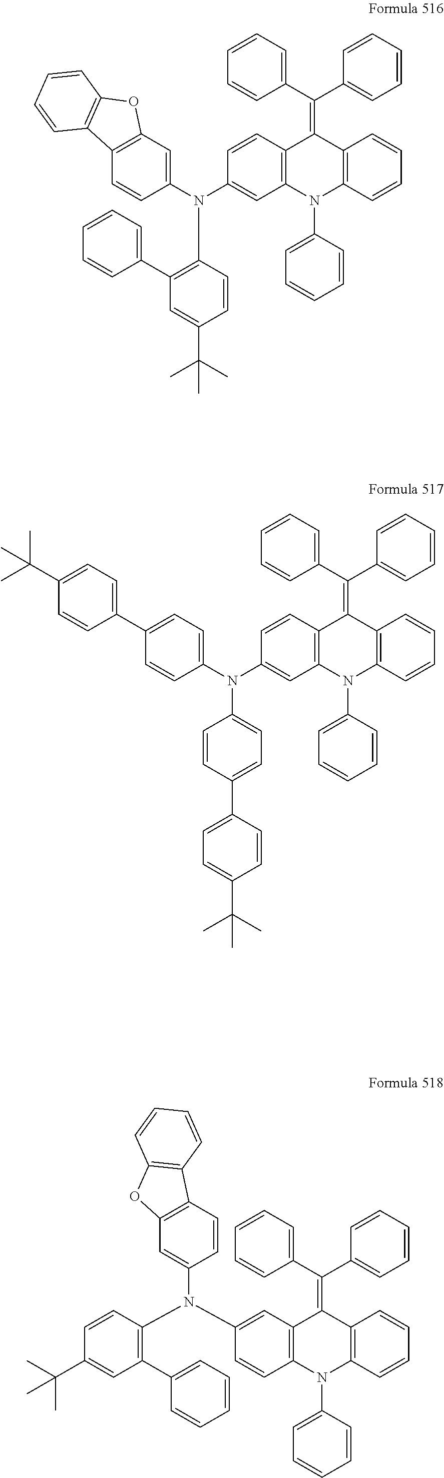

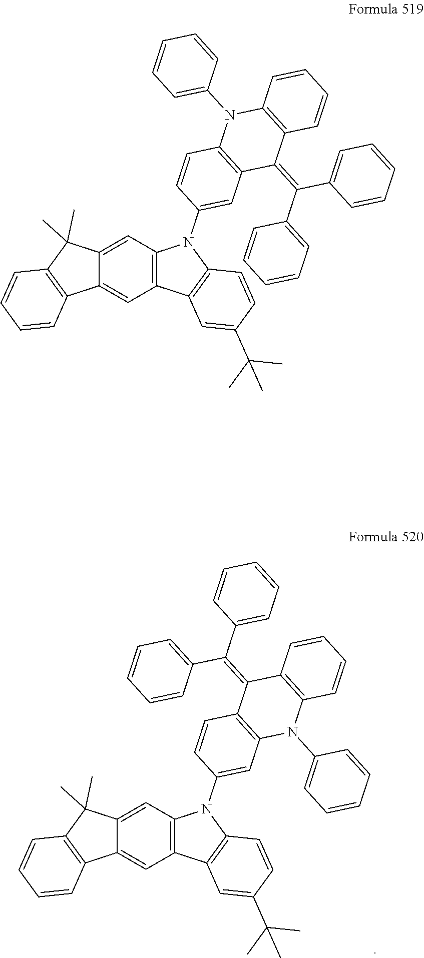

In order to achieve the above technical objectives, on the other hand, an embodiment of the present application discloses an application of the above tetrastyrene-based compound as an electroluminescent organic material in an electronic device.

In order to achieve the above-mentioned technical objectives, in another aspect, an embodiment of the present application discloses an electronic device, including a substrate, an anode, a cathode, and one or more organic material layers interposed between the anode and the cathode, and at least one of the one or more organic material layers includes the above-mentioned tetrastyrene-based compound.

Further, as for the electronic device, the organic material layer includes a hole injection layer, a hole transport layer, an electron injection layer, and an electron transport layer, and a light-emitting layer.

Beneficial effects of the present application are: embodiments of the present application provide a tetrastyrene-based compound, an application thereof, and an electronic device using the same, wherein the tetrastyrene-based compound includes an aromatic amine and a rigid tetrastyrene structure, the aromatic amine can effectively improve the hole injection and transport performance, thereby improving the balance of electrons and holes of the organic light-emitting diode, achieving a lower voltage and higher efficiency; and the rigid tetrastyrene structure is conducive to the formation of evaporation materials of melting type, thereby being conducive to the stability of mass production evaporation. Such materials can achieve high-efficiency preparation of electroluminescent devices, and can be used in the manufacture of display devices.

### DETAILED DESCRIPTION OF PREFERRED EMBODIMENTS

Details of the tetrastyrene-based compound and electronic device using the same provided in the embodiments of the present application is explained and described as follows.

The general structural formula of the tetrastyrene-based compound provided in an embodiment of the present application is shown in the following formula 1:

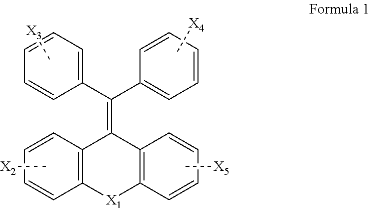

In Formula 1, X1 is selected from,

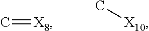

### O, N-X₁₁ or S;

X2, X3, X4, and X5 are each independently selected from an arylamine with a general structural formula as shown in Formula 2, a hydrogen, an alkyl group having 1 to 22 carbon atoms, an alkoxy group having 1 to 22 carbon atoms, or a heteroalkyl group having 1 to 22 carbon atoms, a mono- or multi-substituted or unsubstituted aryl group, or a substituted or unsubstituted heteroaryl group, wherein a heteroatom of the heteroalkyl group is O, N, F, S, or Si, a heteroatom of the heteroaryl group is Si, Ge, N, P, O, S, or Se, and at least one of X2, X3, X4, and X5 is an arylamine with the general structural formula as shown in Formula 2:

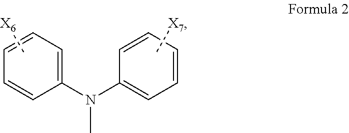

In the above formula, X6, X7, X8, X9, X10, and X11 are each independently selected from hydrogen, an alkyl group, an alkoxy group having 1 to 22 carbon atoms, or a heteroalkyl group having 1 to 22 carbon atoms, a mono- or multi-substituted or unsubstituted aryl group, or a substituted or unsubstituted heteroaryl group, wherein a heteroatom of the heteroalkyl group is O, N, F, S, or Si, and a heteroatom of the heteroaryl group is Si, Ge, N, P, O, S, or Se, or at least two of X2, X3, X4, X5, X6, X7, X8, X9, X10, and X11 are connected to each other to form a monocyclic ring or fused ring of an aromatic ring or a heterocyclic ring when the at least two of X2, X3, X4, X5, X6, X7, X8, X9, X10, and X11 are adjacent aryl groups or heteroaryl groups; and
when expressed as “substituted or unsubstituted”, optional substituents of each of the aryl group and the heteroaryl group are each independently selected from H, halogen, —OH, —SH, —CN, —NO₂, and an alkylthio group having 1 to 15 carbon atoms, an alkyl group having 1 to 40 carbon atoms, or a substituted alkyl group having 1 to 40 carbon atoms.

It can be seen that the tetrastyrene-based compound of the above embodiment contains a rigid tetrastyrene structure and an aromatic amine, wherein the aromatic amine can effectively improve the hole injection and transport performance, thereby improving the balance of electrons and holes of the organic light-emitting diode; and the rigid tetrastyrene structure is conducive to the formation of evaporation materials of melting type, thereby being conducive to the stability of mass production evaporation.

In a preferred embodiment of the present application, a general structural formula of X1 can be

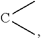

and a general structural formula of the tetrastyrene-type compound can be as shown in the following Formula 3:

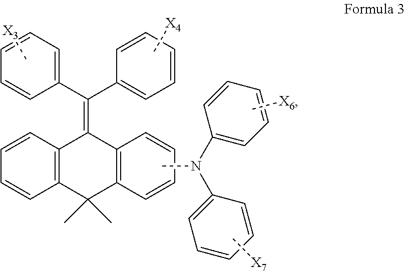

In Formula, X3, X4, X6, and X7 are each independently selected from a mono- or multi-substituted, or unsubstituted aryl group, or a substituted or unsubstituted heteroaryl group, or at least two of X3, X4, X6, and X7 are connected to each other to form a monocyclic ring or fused ring of an aromatic ring or a heterocyclic ring when the at least two of X3, X4, X6, and X7 are adjacent aryl groups or heteroaryl groups.

Furthermore, a structure of the tetrastyrene-based compound is represented by any one of the following general Formulas 301-320:

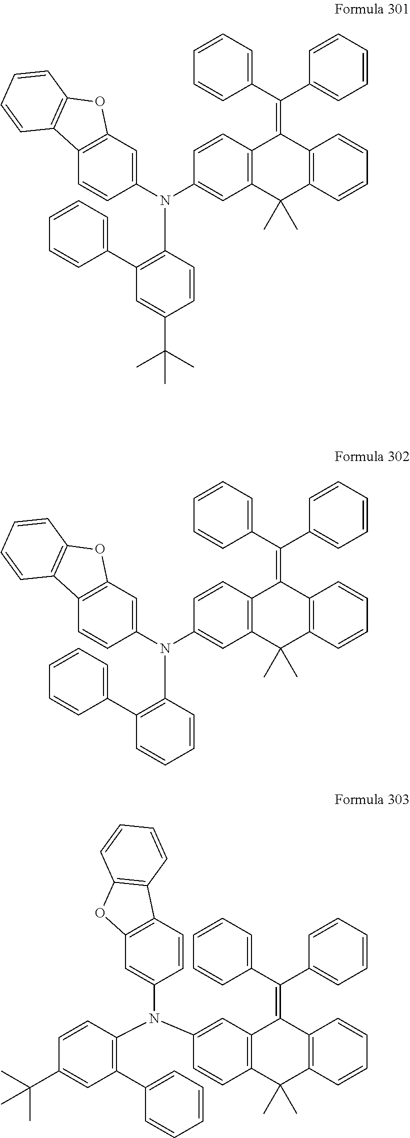

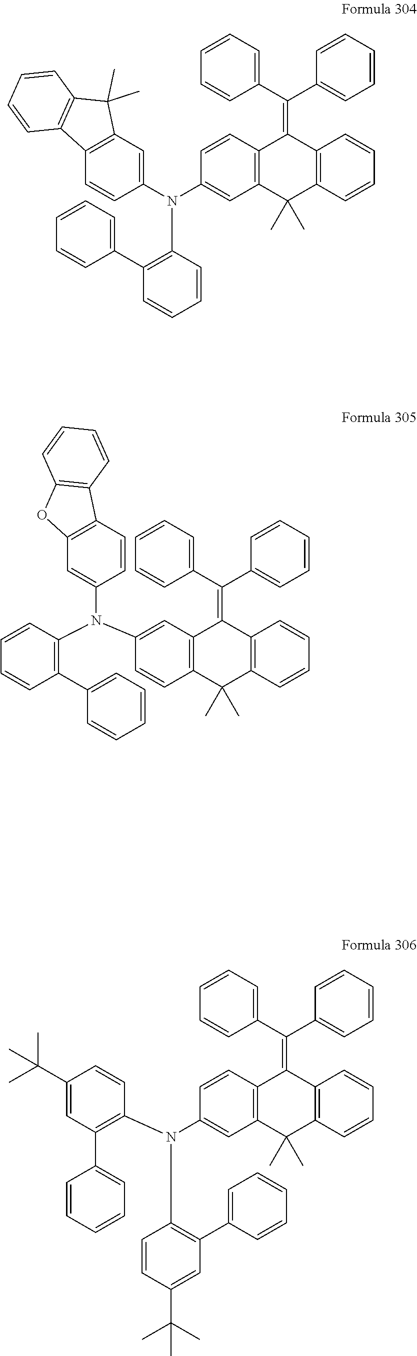

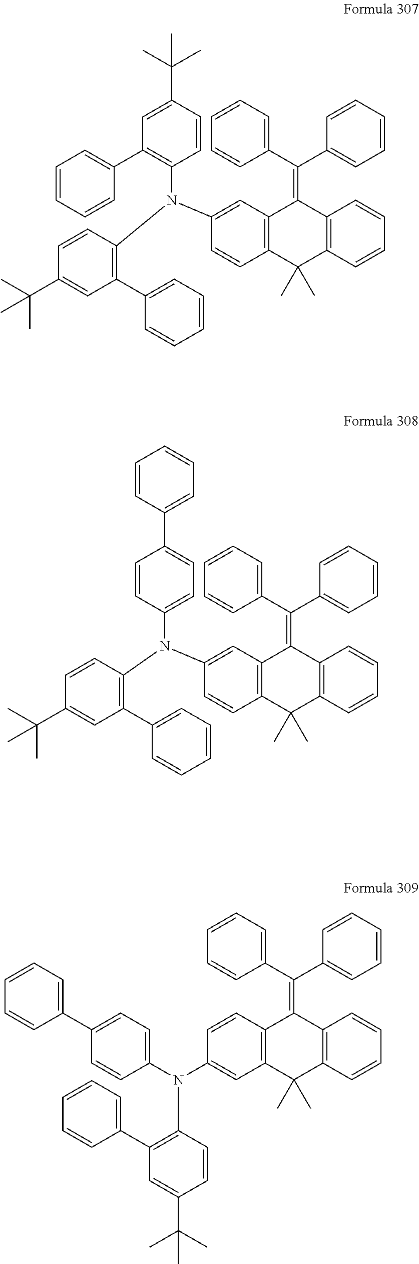

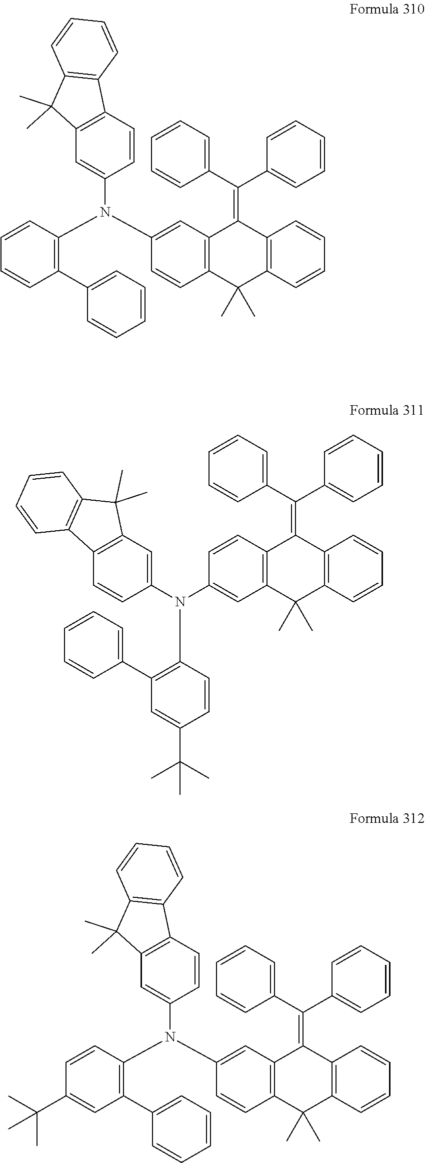

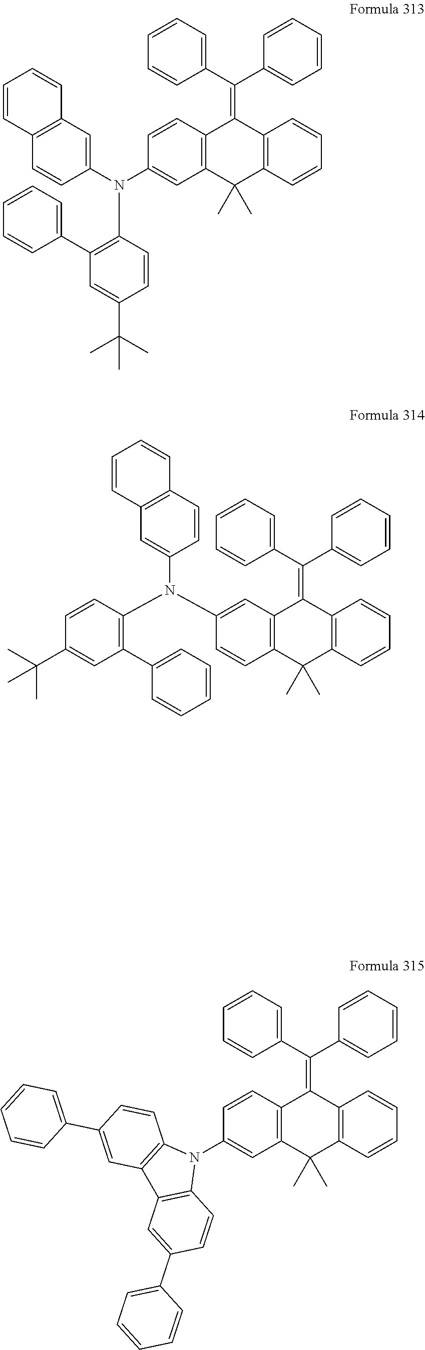

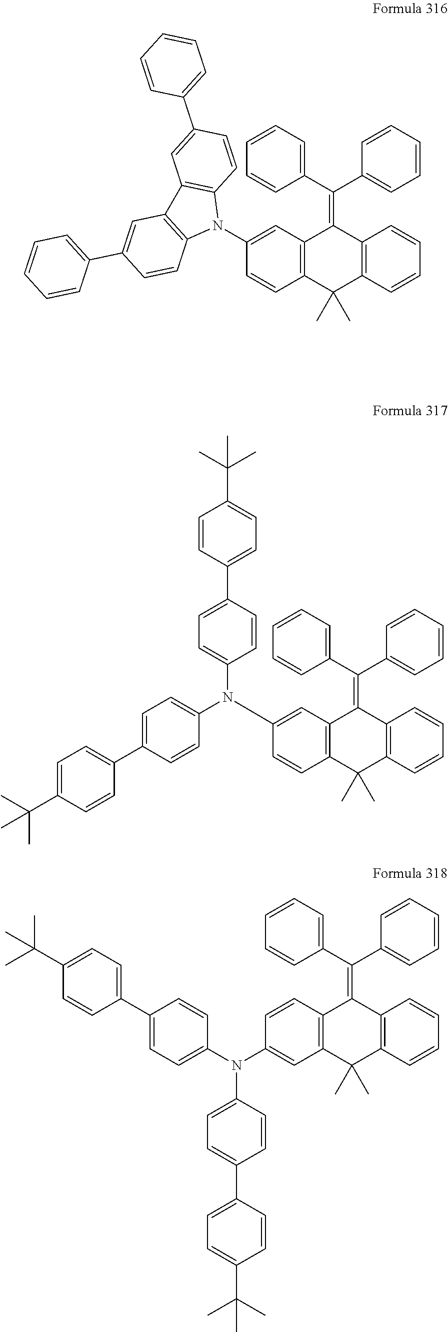

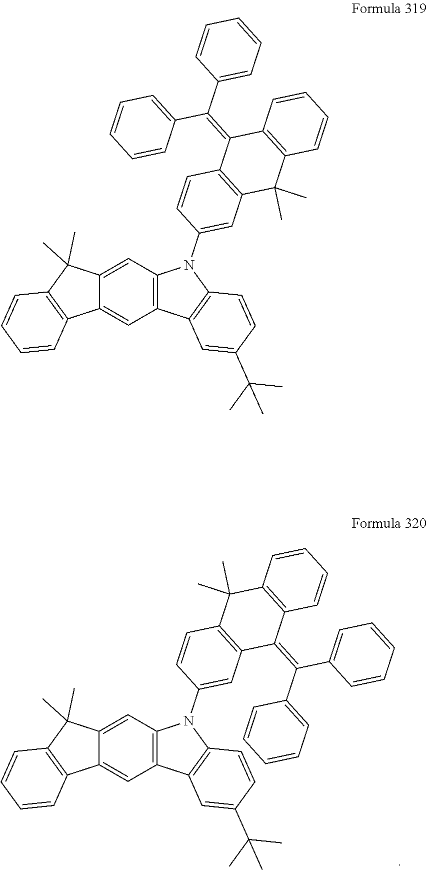

In another preferred embodiment of the present application, a general structural formula of X1 is O, and a general structural formula of the tetrastyrene-based compound is shown in the following Formula 4:

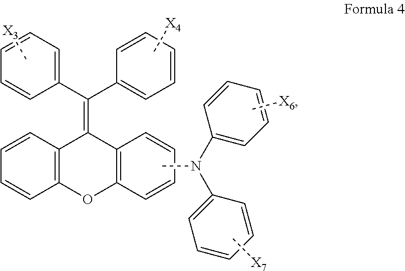

In Formula 4, X3, X4, X6, and X7 are each independently selected from a mono- or multi-substituted, or unsubstituted aryl group, or a substituted or unsubstituted heteroaryl group, or at least two of X3, X4, X6, and X7 are connected to each other to form a monocyclic ring or fused ring of an aromatic ring or a heterocyclic ring when the at least two of X3, X4, X6, and X7 are adjacent aryl groups or heteroaryl groups.

Furthermore, a structure of the tetrastyrene-based compound is represented by any one of the following general Formulas 401-420:

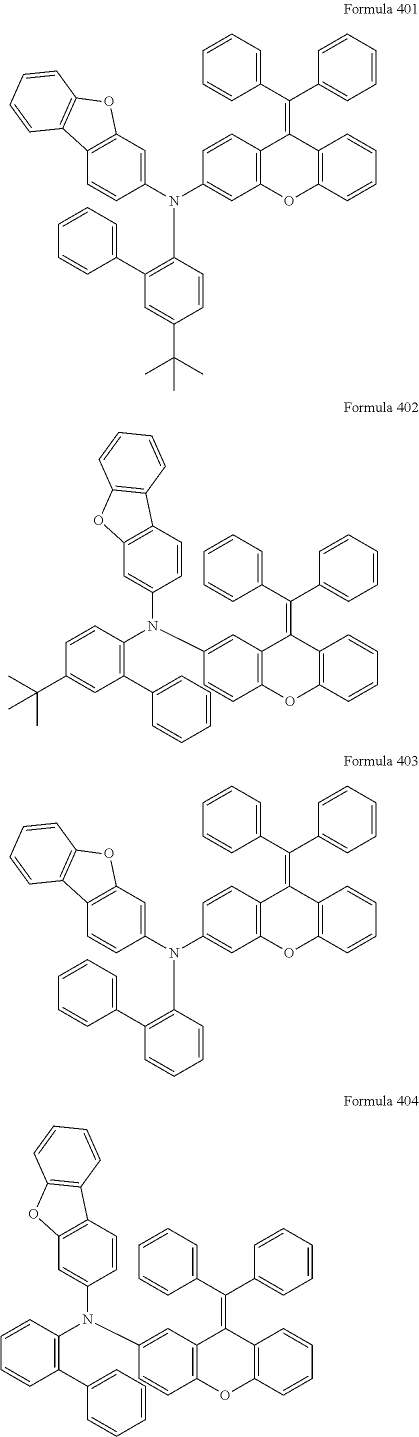

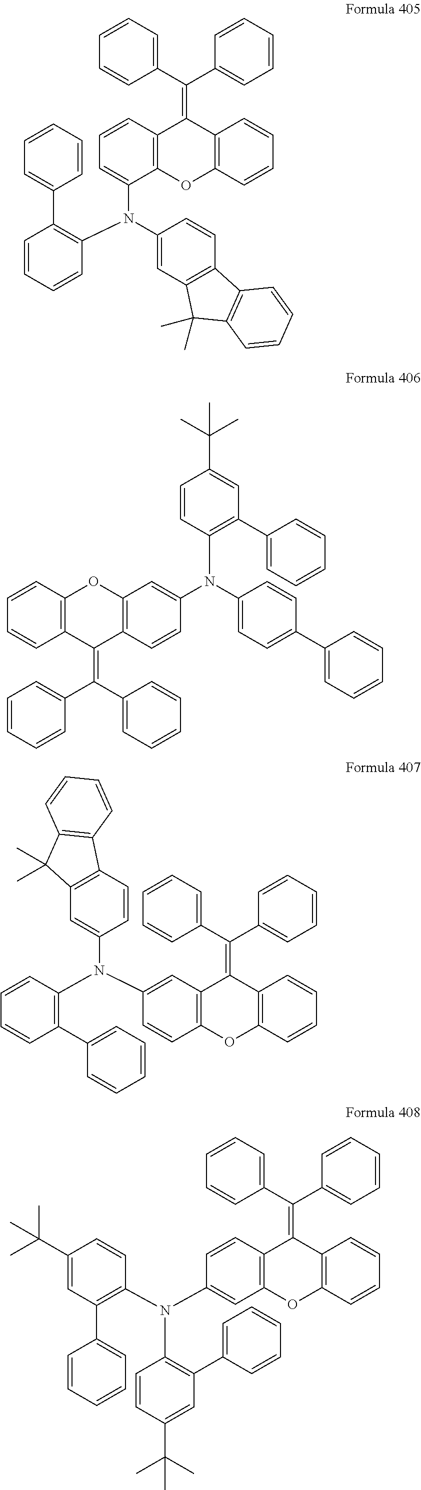

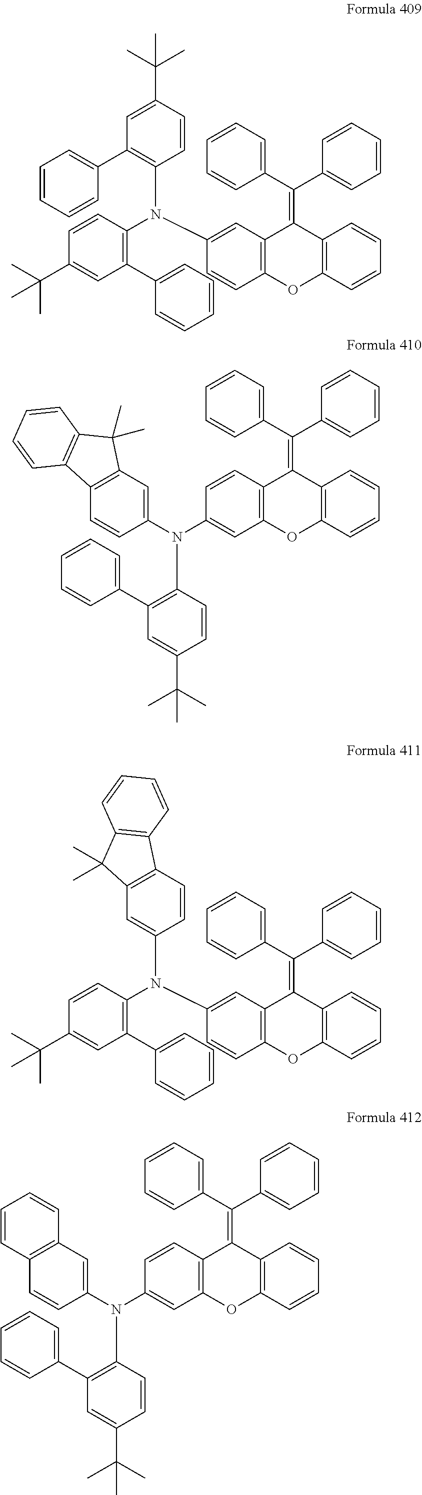

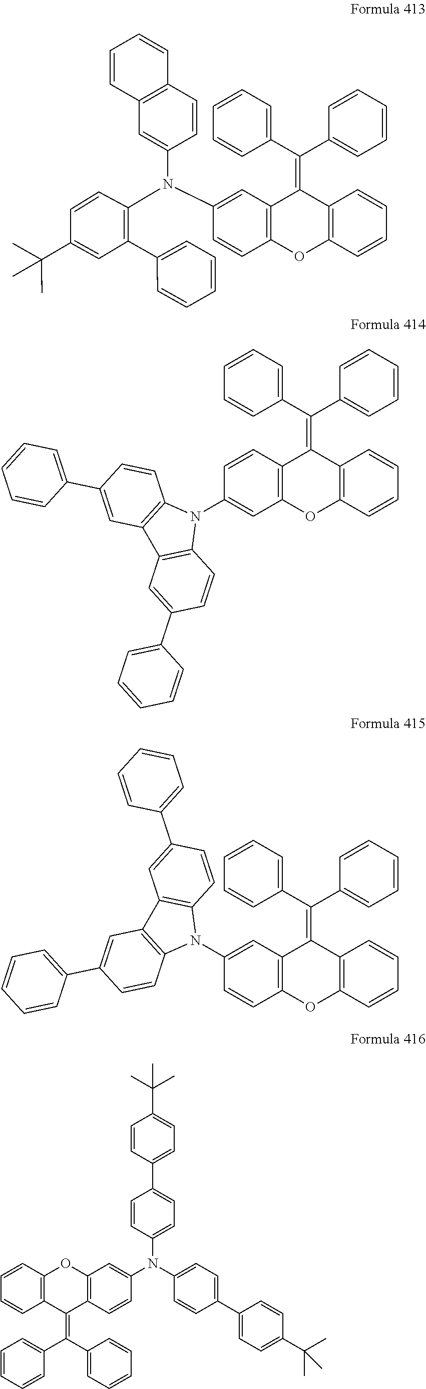

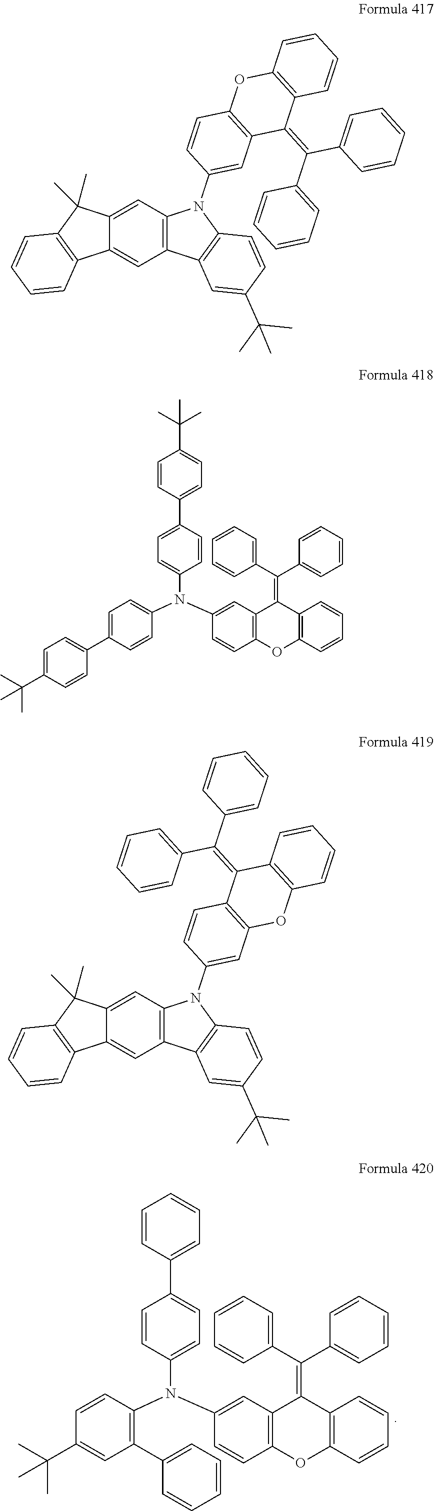

In further another preferred embodiment of the present application, a general structural formula of X1 is N-ph, and a general structural formula of the tetrastyrene-based compound is shown in Formula 5 below:

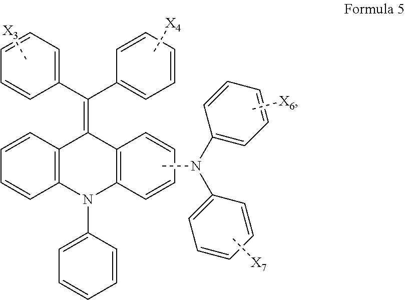

In Formula 5, X3, X4, X6, and X7 are each independently selected from a mono- or multi-substituted, or unsubstituted aryl group, or a substituted or unsubstituted heteroaryl group, or at least two of X3, X4, X6, and X7 are connected to each other to form a monocyclic ring or fused ring of an aromatic ring or a heterocyclic ring when the at least two of X3, X4, X6, and X7 are adjacent aryl groups or heteroaryl groups.

Furthermore, a structure of the tetrastyrene-based compound is represented by any one of the following general Formulas 501-520:

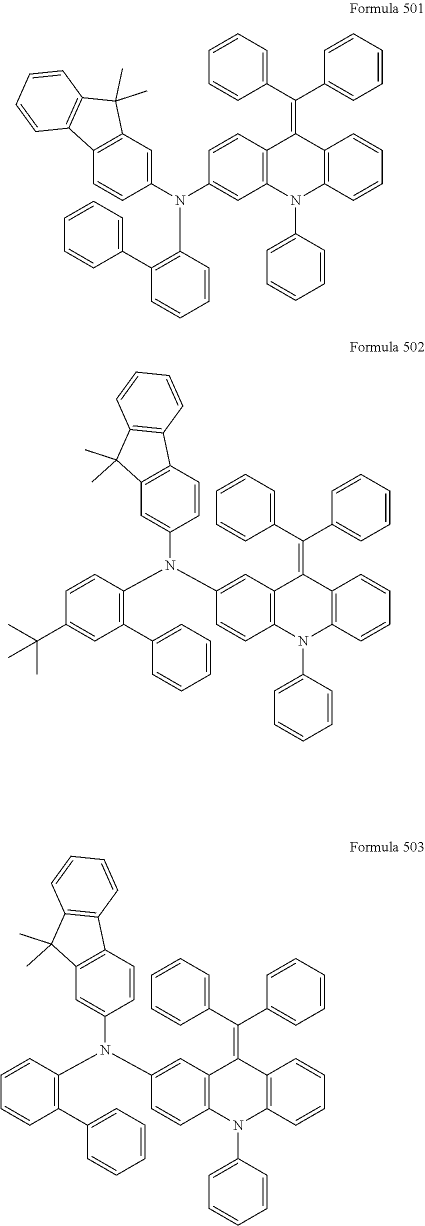

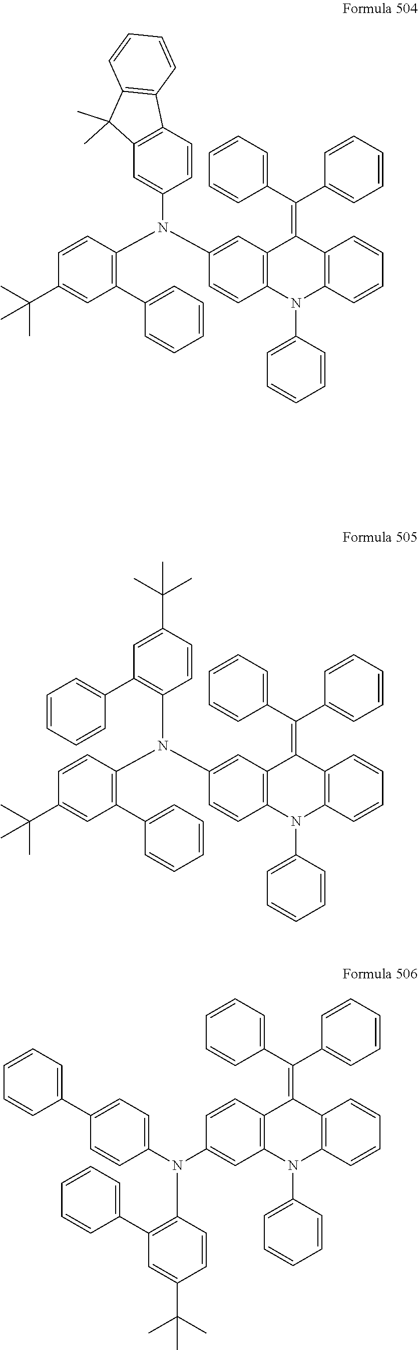

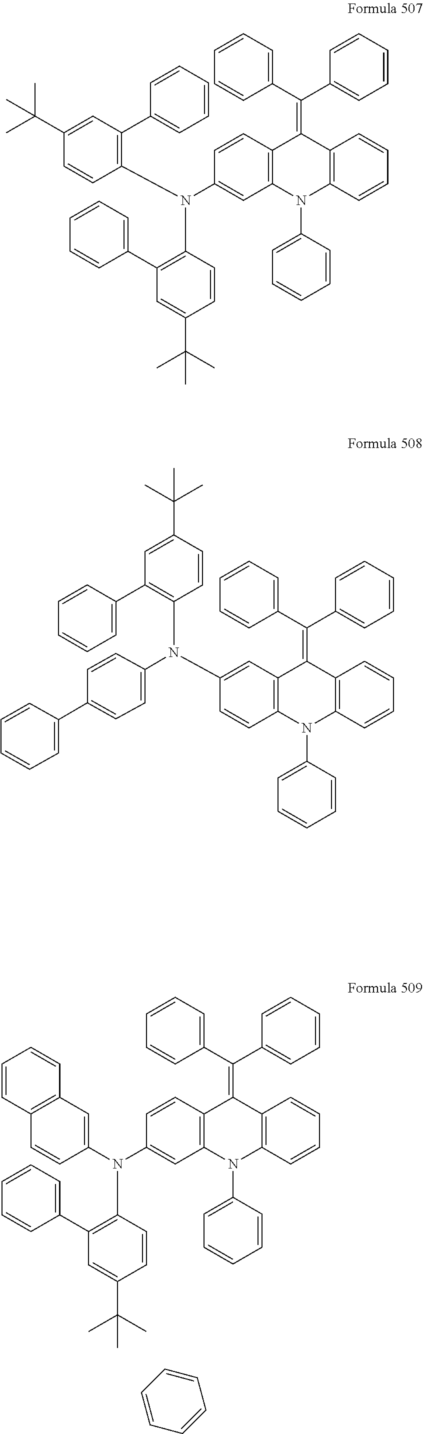

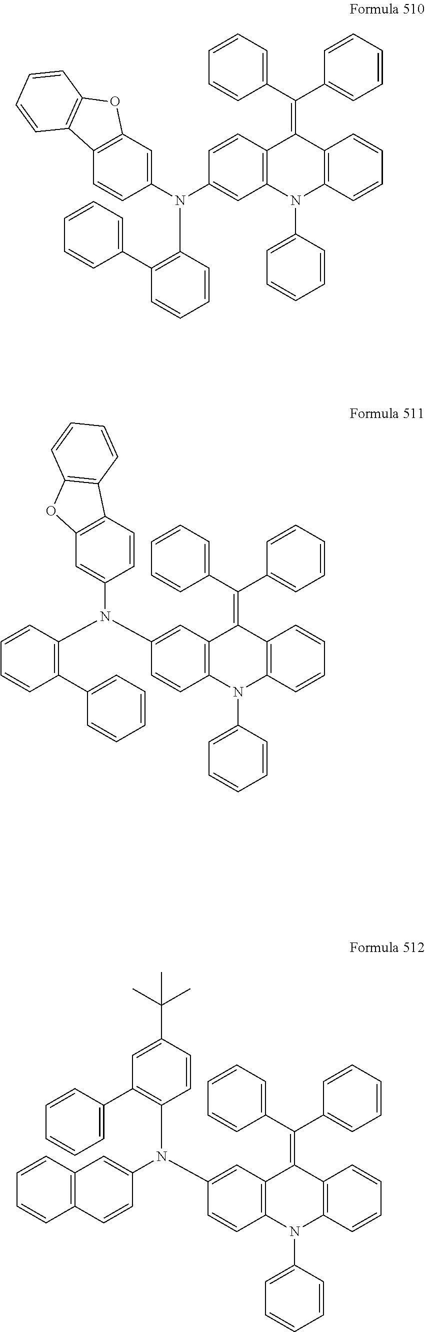

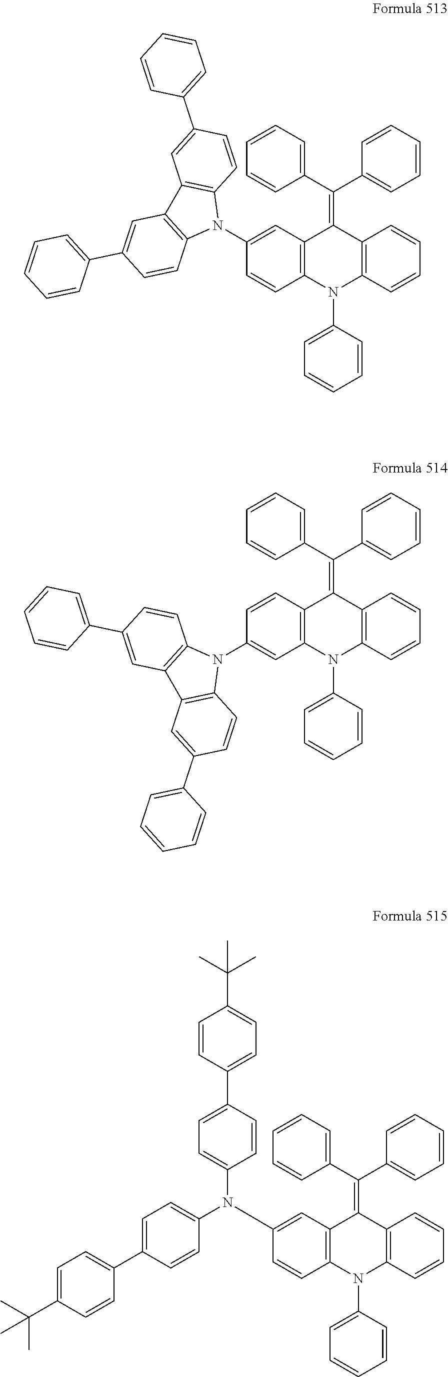

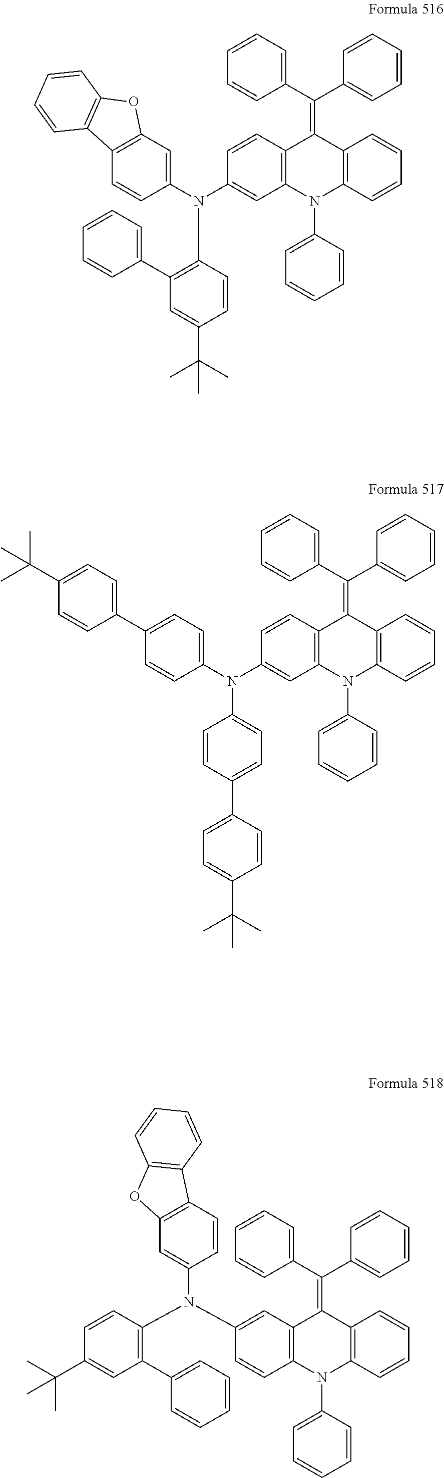

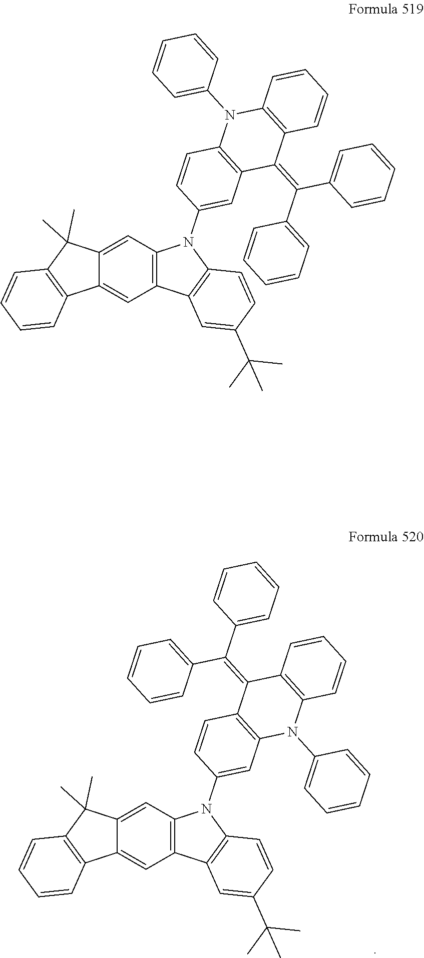

Another embodiment of the present application provides an application of the above-mentioned tetrastyrene-based compound as an electroluminescent organic material in an electronic device.

Still another embodiment of the present application provides an electronic device including a substrate, an anode, a cathode, and one or more organic material layers interposed between the anode and the cathode, wherein at least one of the one or more organic material layers containing the tetrastyrene-based compound of the above-mentioned embodiments, wherein the organic material layer may include a hole injection layer, a hole transport layer, an electron injection layer, an electron transport layer, and a light-emitting layer. Here, the electronic device may be an electroluminescent device, such as an organic light-emitting diode device.

Hereinafter, methods of preparing the tetrastyrene-based compound provided in the above embodiments of the present application will be described.

### EXAMPLE 1

The synthesis reaction of the tetrastyrene-based compound of target structure 1 is shown in Scheme 6:

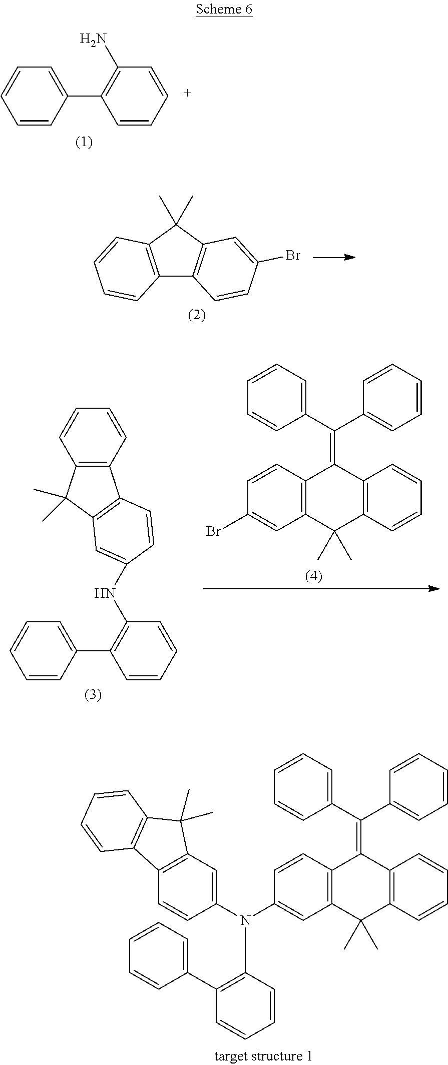 [SMILES: Nc1ccccc1-c1ccccc1 ; CC1(C)c2ccccc2-c2ccc(Br)cc21 ; CC1(C)c2ccccc2-c2ccc(Nc3ccccc3-c3ccccc3)cc21 ; CC1(C)c2ccccc2C(=C(c2ccccc2)c2ccccc2)c2ccc(Br)cc21 ; CC1(C)c2ccccc2C(=C(c2ccccc2)c2ccccc2)c2ccc(N(c3ccc4c(c3)C(C)(C)c3ccccc3-4)c3ccccc3-c3ccccc3)cc21]

The method of preparing the tetrastyrene-based compound of the target structure 1 provided in Example 1 is as follows:

Compound (1) (6 mmol, 1.02 g), Compound (2) (5 mmol, 1.37 g), CuI (0.3 mmol, 0.06 g), potassium carbonate (K₂CO_{3,} 5 mmol, 0.69 g), and 50mL of 1,4-dioxane were added into a 100 mL two-necked bottle, and stirred and heated to 100° C. under argon atmosphere for a reaction for 12 hour, which was then dissolved with dichloromethane (300 mL) and added with saturated NH₄Cl (200 mL) solution, extracted by dichloromethane, dried by the organic phase with anhydrous sodium sulfate, and concentrated, followed by column separation, wherein 200-300 mesh silica gel was used as a stationary phase, and dichloromethane was used as an eluent, to obtain an intermediate, 1.64 g of Compound (3), with a yield of 91%.

The product, Compound (3) (4.6 mmol, 1.64 g), from the previous steps, Compound (4) (5.0 mmol, 2.26 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 2.15 g of white powdery target structure 1, with a yield of 64%. Matrix-assisted laser desorption ionization time-of-flight mass spectrometer (MALDI-TOF): calculated value m/z, 731.98; measured value m/z, 731.12. Elemental analysis (EA): calculated value: carbon C, 91.89; hydrogen H, 6.20; nitrogen N, 1.91; measured value: carbon C, 91.79; hydrogen H, 6.43; nitrogen N, 1.78.

### EXAMPLE 2

The synthesis reaction of the tetrastyrene-based compound of target structure 2 is shown in Scheme 7:

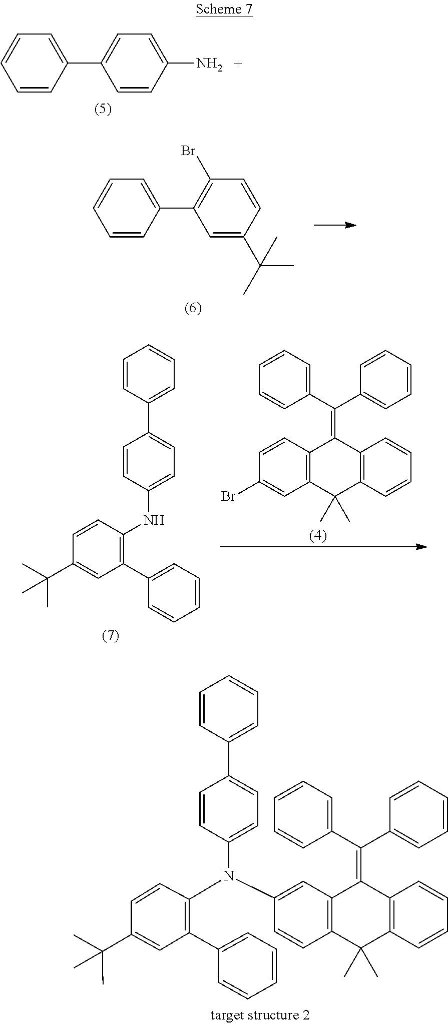 [SMILES: Nc1ccc(-c2ccccc2)cc1 ; CC(C)(C)c1ccc(Br)c(-c2ccccc2)c1 ; CC(C)(C)c1ccc(Nc2ccc(-c3ccccc3)cc2)c(-c2ccccc2)c1 ; CC1(C)c2ccccc2C(=C(c2ccccc2)c2ccccc2)c2ccc(Br)cc21 ; CC(C)(C)c1ccc(N(c2ccc(-c3ccccc3)cc2)c2ccc3c(c2)C(=C(c2ccccc2)c2ccccc2)c2ccccc2C3(C)C)c(-c2ccccc2)c1]

The method of preparing the tetrastyrene-based compound of the target structure 2 provided in Example 2 is as follows:

Compound (5) (6 mmol, 1.02 g), Compound (6) (5 mmol, 1.45 g), CuI (0.3 mmol, 0.06 g), potassium carbonate (K₂CO₃, 5 mmol, 0.69 g), and 50 mL of 1,4-dioxane were added into a 100 mL two-necked bottle, and stirred and heated to 100° C. under argon atmosphere for a reaction for 12 hour, which was then dissolved with dichloromethane (300 mL) and added with saturated NH₄Cl (200 mL) solution, extracted by dichloromethane, dried by the organic phase with anhydrous sodium sulfate, and concentrated, followed by column separation, wherein 200-300 mesh silica gel was used as a stationary phase, and dichloromethane was used as an eluent, to obtain an intermediate, 1.67 g of Compound (7), with a yield of 85%.

The product, Compound (7) (4.3 mmol, 1.67 g), from the previous steps, Compound (4) (5.0 mmol, 2.26 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 2.41 g of white powdery target structure 2, with a yield of 75%. Matrix-assisted laser desorption ionization time-of-flight mass spectrometer (MALDI-TOF): calculated value m/z, 748.03; measured value m/z, 748.57. Elemental analysis (EA): calculated value: carbon C, 91.52; hydrogen H, 6.60; nitrogen N, 1.87; measured value: carbon C, 91.74; hydrogen H, 6.53; nitrogen N, 1.73.

### EXAMPLE 3

The synthesis reaction of the tetrastyrene-based compound of the target structure 3 is shown in the Scheme 8:

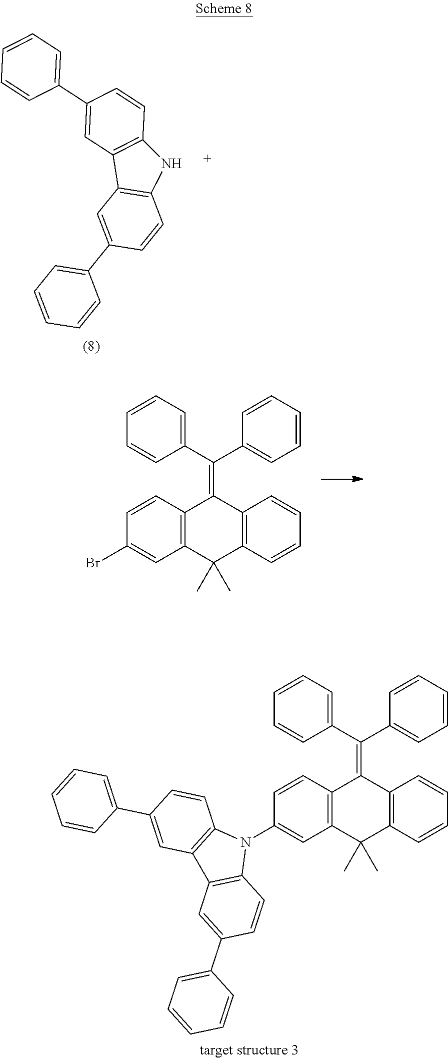 [SMILES: c1ccc(-c2ccc3[nH]c4ccc(-c5ccccc5)cc4c3c2)cc1 ; CC1(C)c2ccccc2C(=C(c2ccccc2)c2ccccc2)c2ccc(Br)cc21 ; CC1(C)c2ccccc2C(=C(c2ccccc2)c2ccccc2)c2ccc(-n3c4ccc(-c5ccccc5)cc4c4cc(-c5ccccc5)ccc43)cc21]

The method of preparing the tetrastyrene-based compound of the target structure 3 provided in Example 3 is as follows:

Compound (8) (5 mmol, 1.60 g), Compound (4) (5.0 mmol, 2.48 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 2.70 g of white powdery target structure 3, with a yield of 80%. Matrix-assisted laser desorption ionization time-of-flight mass spectrometer (MALDI-TOF): calculated value m/z, 674.87; measured value m/z, 674.35. Elemental analysis (EA): calculated value: carbon C, 92.55; hydrogen H, 5.38; nitrogen N, 2.08; measured value: carbon C, 92.10; hydrogen H, 5.56; nitrogen N, 2.34.

### EXAMPLE 4

The synthesis reaction of the tetrastyrene-based compound of target structure 4 is shown in Scheme 9:

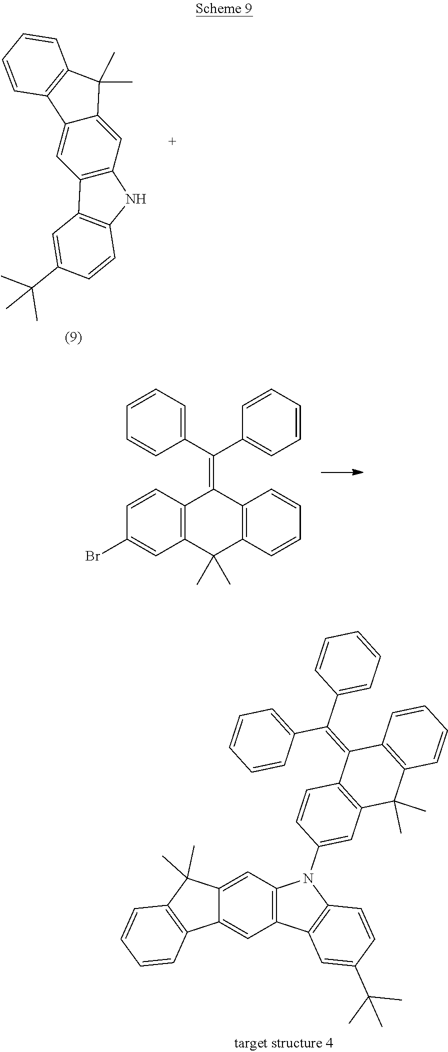 [SMILES: CC(C)(C)c1ccc2[nH]c3cc4c(cc3c2c1)-c1ccccc1C4(C)C ; CC1(C)c2ccccc2C(=C(c2ccccc2)c2ccccc2)c2ccc(Br)cc21 ; CC(C)(C)c1ccc2c(c1)c1cc3c(cc1n2-c1ccc2c(c1)C(C)(C)c1ccccc1C2=C(c1ccccc1)c1ccccc1)C(C)(C)c1ccccc1-3]

The method of preparing the tetrastyrene-based compound of the target structure 4 provided in Example 4 is as follows:

Compound (9) (5mmol, 1.70 g), Compound (4) (5.0 mmol, 2.48 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 2.55 g of white powdery target structure 4, with a yield of 72%. Matrix-assisted laser desorption ionization time-of-flight mass spectrometer (MALDI-TOF): calculated value m/z, 709.38; measured value m/z, 709.11. Elemental analysis (EA): calculated value: carbon C, 91.35; hydrogen H, 6.67; nitrogen N, 1.97; measured value: C, 91.57; H, 6.03; N, 2.40.

### EXAMPLE 5

The synthesis reaction of the tetrastyrene-based compound of target structure 5 is shown in Scheme 10:

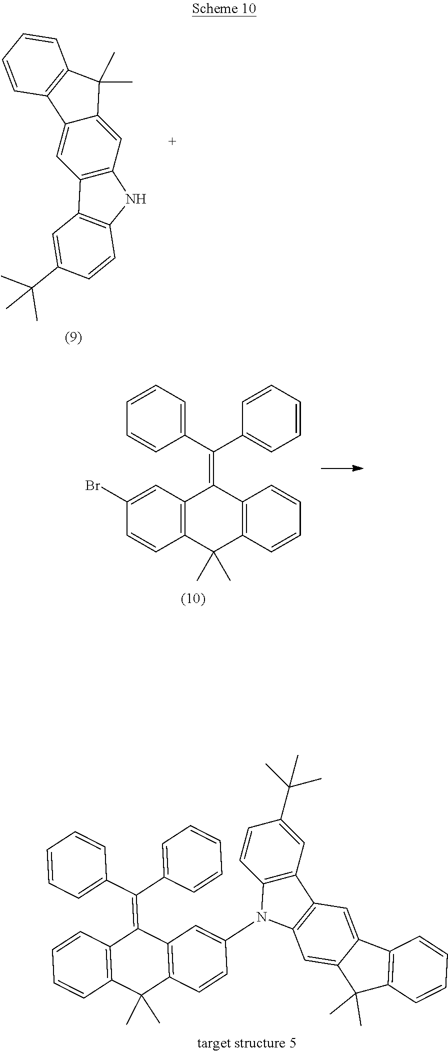 [SMILES: CC(C)(C)c1ccc2[nH]c3cc4c(cc3c2c1)-c1ccccc1C4(C)C ; CC1(C)c2ccccc2C(=C(c2ccccc2)c2ccccc2)c2cc(Br)ccc21 ; CC(C)(C)c1ccc2c(c1)c1cc3c(cc1n2-c1ccc2c(c1)C(=C(c1ccccc1)c1ccccc1)c1ccccc1C2(C)C)C(C)(C)c1ccccc1-3]

The method of preparing the tetrastyrene-based compound of the target structure 5 provided in Example 5 is as follows:

Compound (9) (5mmol, 1.70 g), Compound (10) (5.5 mmol, 2.48 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 2.31 g of white powdery target structure 5 with a yield of 65%. Matrix-assisted laser desorption ionization time-of-flight mass spectrometer (MALDI-TOF): calculated value m/z, 709.38; measured value m/z, 709.15. Elemental analysis method (EA): calculated value: carbon C, 91.35; hydrogen H, 6.67; nitrogen N, 1.97; measured value: C, 91.41; H, 6.53; N, 2.06.

### EXAMPLE 6

The synthesis reaction of the tetrastyrene-based compound of target structure 6 is shown in Scheme 11:

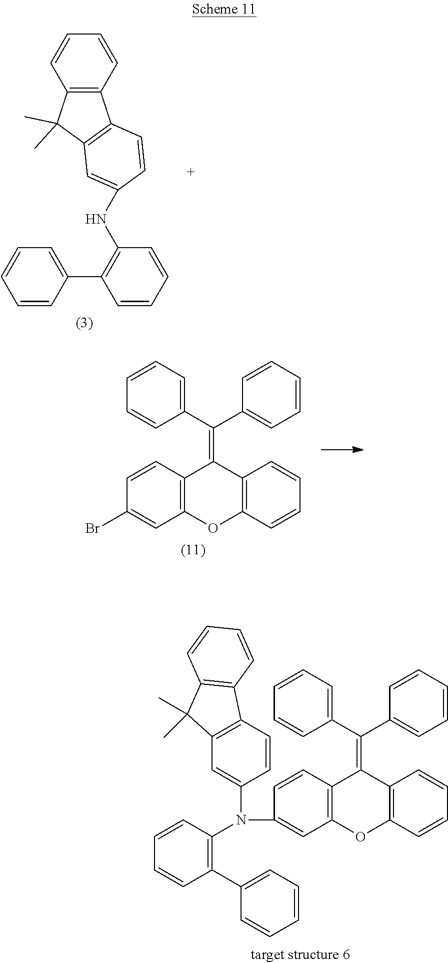 [SMILES: CC1(C)c2ccccc2-c2ccc(Nc3ccccc3-c3ccccc3)cc21 ; Brc1ccc2c(c1)Oc1ccccc1C2=C(c1ccccc1)c1ccccc1 ; CC1(C)c2ccccc2-c2ccc(N(c3ccc4c(c3)Oc3ccccc3C4=C(c3ccccc3)c3ccccc3)c3ccccc3-c3ccccc3)cc21]

The method of preparing the tetrastyrene-based compound of the target structure 6 provided in Example 6 is as follows:

Compound (3) (4.0 mmol, 1.45 g), Compound (11) (5.0 mmol, 2.13 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 2.20 g of white powdery target structure 6, with a yield of 78%. Matrix-assisted laser desorption ionization time-of-flight mass spectrometer (MALDI-TOF): calculated value m/z, 705.94; measured value m/z, 705.19. Elemental analysis (EA): calculated value: carbon C, 91.18; hydrogen H, 5.57; nitrogen N, 1.98; measured value: C, 91.32; H, 5.71; N, 1.84.

### EXAMPLE 7

The synthesis reaction of the tetrastyrene-based compound of target structure 7 is shown in Scheme 12:

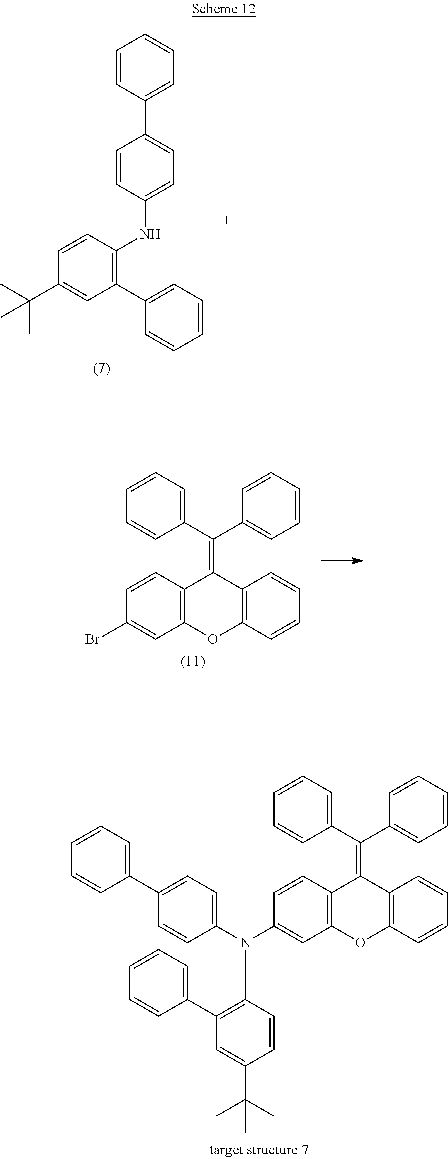 [SMILES: CC(C)(C)c1ccc(Nc2ccc(-c3ccccc3)cc2)c(-c2ccccc2)c1 ; Brc1ccc2c(c1)Oc1ccccc1C2=C(c1ccccc1)c1ccccc1 ; CC(C)(C)c1ccc(N(c2ccc(-c3ccccc3)cc2)c2ccc3c(c2)Oc2ccccc2C3=C(c2ccccc2)c2ccccc2)c(-c2ccccc2)c1]

The method of preparing the tetrastyrene-based compound of the target structure 7 provided in Example 7 is as follows:

Compound (7) (4.0 mmol, 1.57 g), Compound (11) (5.0 mmol, 2.13 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 1.99 g of white powdery target structure 7 with a yield of 69%. Matrix-assisted laser desorption ionization time-of-flight mass spectrometer (MALDI-TOF): calculated value m/z, 721.94; measured value m/z, 721.87. Elemental analysis (EA): calculated value: carbon C, 89.84; hydrogen H, 6.00; nitrogen N, 1.94; measured value: C, 90.05; H, 5.92; N, 1.99.

### EXAMPLE 8

The synthesis reaction of the tetrastyrene-based compound of target structure 8 is shown in Scheme 13:

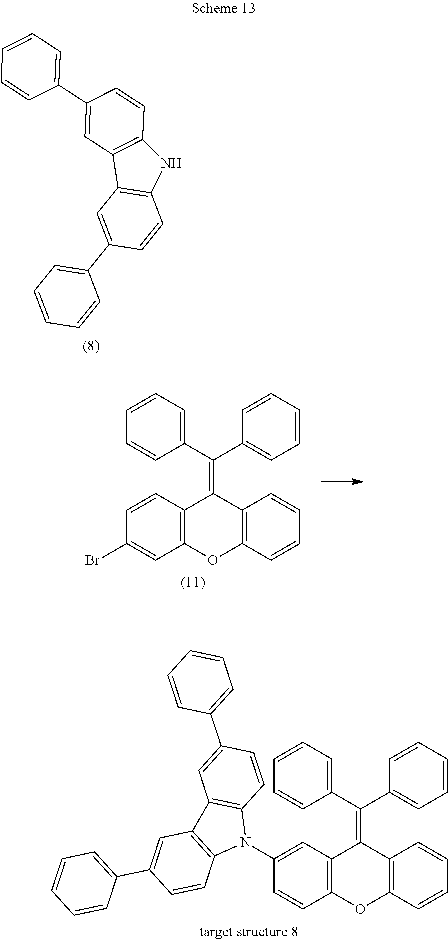 [SMILES: c1ccc(-c2ccc3[nH]c4ccc(-c5ccccc5)cc4c3c2)cc1 ; Brc1ccc2c(c1)Oc1ccccc1C2=C(c1ccccc1)c1ccccc1 ; c1ccc(C(=C2c3ccccc3Oc3ccc(-n4c5ccc(-c6ccccc6)cc5c5cc(-c6ccccc6)ccc54)cc32)c2ccccc2)cc1]

The method of preparing the tetrastyrene-based compound of the target structure 8 provided in Example 8 is as follows:

Compound (8) (4.0 mmol, 1.28 g), Compound (11) (5.0 mmol, 2.13 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 1.86 g of white powdery target structure 8, with a yield of 70%. Matrix-assisted laser desorption ionization time-of-flight mass spectrometer (MALDI-TOF): calculated value m/z, 663.82; measured value m/z, 663.19. Elemental analysis method (EA): calculated value: carbon C, 90.47; hydrogen H, 5.01; nitrogen N, 2.11; measured value: C, 90.13; H, 5.32; N, 2.01.

### EXAMPLE 9

The synthesis reaction of the tetrastyrene-based compound of target structure 9 is shown in Scheme 14:

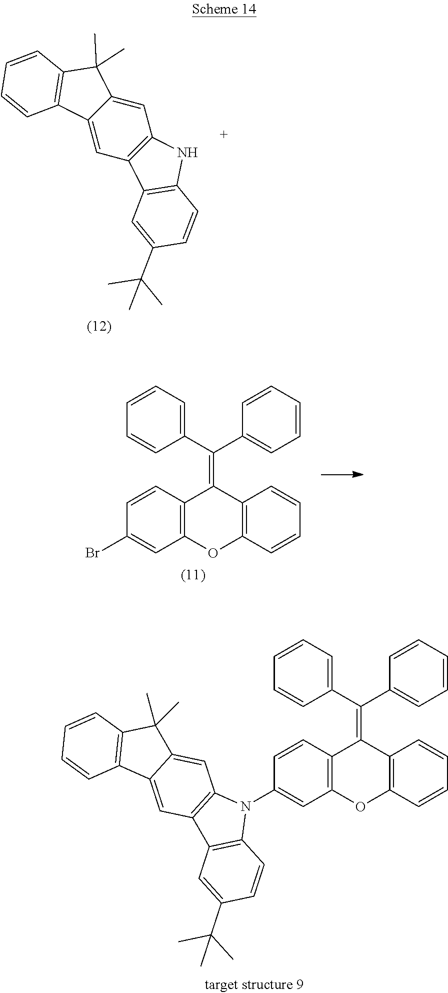 [SMILES: CC(C)(C)c1ccc2[nH]c3cc4c(cc3c2c1)-c1ccccc1C4(C)C ; Brc1ccc2c(c1)Oc1ccccc1C2=C(c1ccccc1)c1ccccc1 ; CC(C)(C)c1ccc2c(c1)c1cc3c(cc1n2-c1ccc2c(c1)Oc1ccccc1C2=C(c1ccccc1)c1ccccc1)C(C)(C)c1ccccc1-3]

The method of preparing the tetrastyrene-based compound of the target structure 9 provided in Example 9 is as follows:

Compound (12) (4.0 mmol, 1.30 g), Compound (11) (5.0 mmol, 2.13 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 2.00 g of white powdery target structure 9, with a yield of 73%. Matrix-assisted laser desorption ionization time-of-flight mass spectrometer (MALDI-TOF): calculated value m/z, 683.9; measured value m/z, 683.53. Elemental analysis method (EA): calculated value: carbon C, 89.57; hydrogen H, 6.04; nitrogen N, 2.05; measured value: C, 89.37; H, 6.21; N, 1.86.

### EXAMPLE 10

The synthesis reaction of the tetrastyrene-based compound of target structure 10 is shown in Scheme 15:

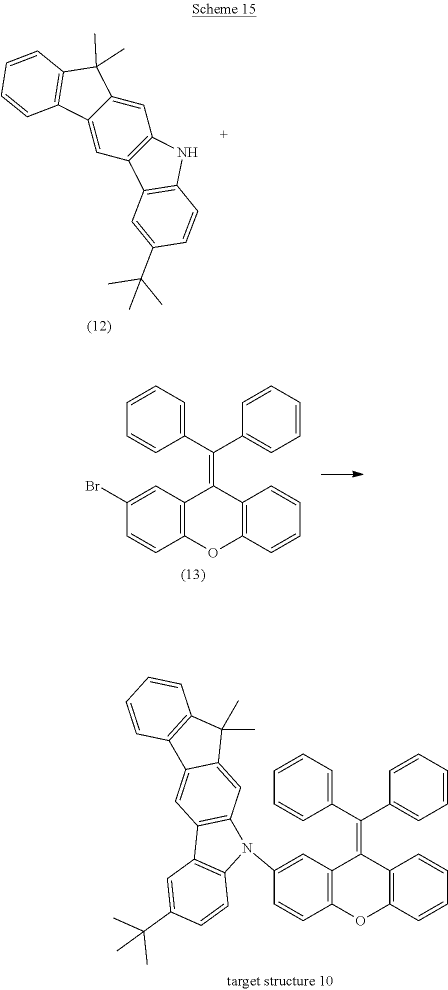 [SMILES: CC(C)(C)c1ccc2[nH]c3cc4c(cc3c2c1)-c1ccccc1C4(C)C ; Brc1ccc2c(c1)C(=C(c1ccccc1)c1ccccc1)c1ccccc1O2 ; CC(C)(C)c1ccc2c(c1)c1cc3c(cc1n2-c1ccc2c(c1)C(=C(c1ccccc1)c1ccccc1)c1ccccc1O2)C(C)(C)c1ccccc1-3]

The method of preparing the tetrastyrene-based compound of the target structure 10 provided in Example 10 is as follows:

Compound (12) (4.0 mmol, 1.30 g), Compound (13) (5.0 mmol, 2.13 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 1.67 g of white powdery target structure 10 with a yield of 61%. Matrix-assisted laser desorption ionization time-of-flight mass spectrometer (MALDI-TOF): calculated value m/z, 683.9; measured value m/z, 683.64. Elemental analysis method (EA): calculated value: carbon C, 89.57; hydrogen H, 6.04; nitrogen N, 2.05; measured value: C, 89.45; H, 6.09; N, 2.14.

### EXAMPLE 11

The synthesis reaction of the tetrastyrene-based compound of target structure 11 is shown in Scheme 16:

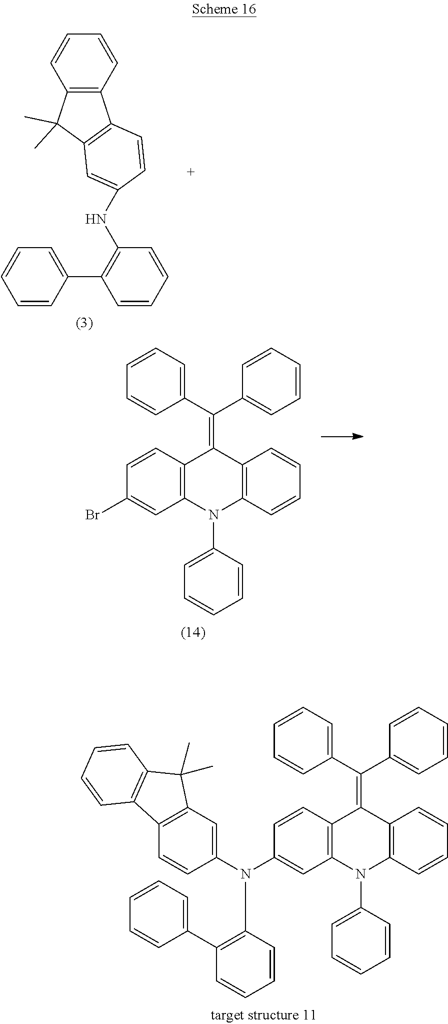

The method of preparing the tetrastyrene-based compound of the target structure 11 provided in Example 11 is as follows:

Compound (3) (4.6 mmol, 1.64 g), Compound (14) (5.0 mmol, 2.50 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 2.28 g of white powdery target structure 11 with a yield of 73%. Matrix-assisted laser desorption ionization time-of-flight mass spectrometer (MALDI-TOF): calculated value m/z, 781.02; measured value m/z, 780.93. Elemental analysis (EA): calculated value carbon: C, 90.73; hydrogen H, 5.68; nitrogen N, 3.59; measured value: C, 90.85; H, 5.72; N, 3.43.

### EXAMPLE 12

The synthesis reaction of the tetrastyrene-based compound of the target structure 12 is shown in the Scheme 17:

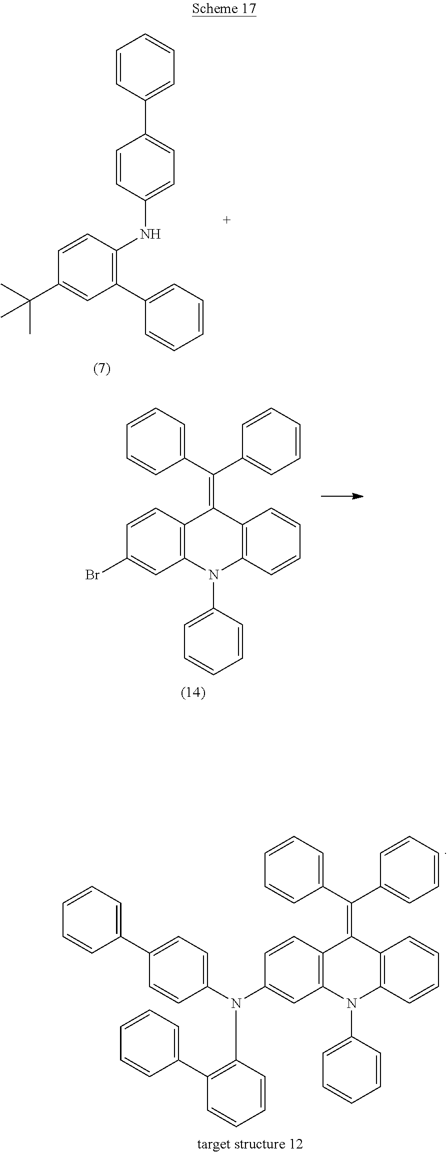 [SMILES: CC(C)(C)c1ccc(Nc2ccc(-c3ccccc3)cc2)c(-c2ccccc2)c1 ; Brc1ccc2c(c1)N(c1ccccc1)c1ccccc1C2=C(c1ccccc1)c1ccccc1 ; c1ccc(C(=C2c3ccccc3N(c3ccccc3)c3cc(N(c4ccc(-c5ccccc5)cc4)c4ccccc4-c4ccccc4)ccc32)c2ccccc2)cc1]

The method of preparing the tetrastyrene-based compound of the target structure 12 provided in Example 12 is as follows:

Compound (7) (4.0mmol, 1.57 g), Compound (14) (5.0 mmol, 2.50 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 2.07 g of white powdery target structure 12 with a yield of 65%. Matrix-assisted laser desorption ionization time-of-flight mass spectrometer (MALDI-TOF): calculated value m/z, 797.06; measured value m/z, 797.53. Elemental analysis method (EA): calculated value: carbon C, 90.42; hydrogen H, 6.07; nitrogen N, 3.51; measured value: C, 90.35; H, 5.98; N, 3.67.

### EXAMPLE 13

The synthesis reaction of the tetrastyrene-based compound of target structure 13 is shown in Scheme 18:

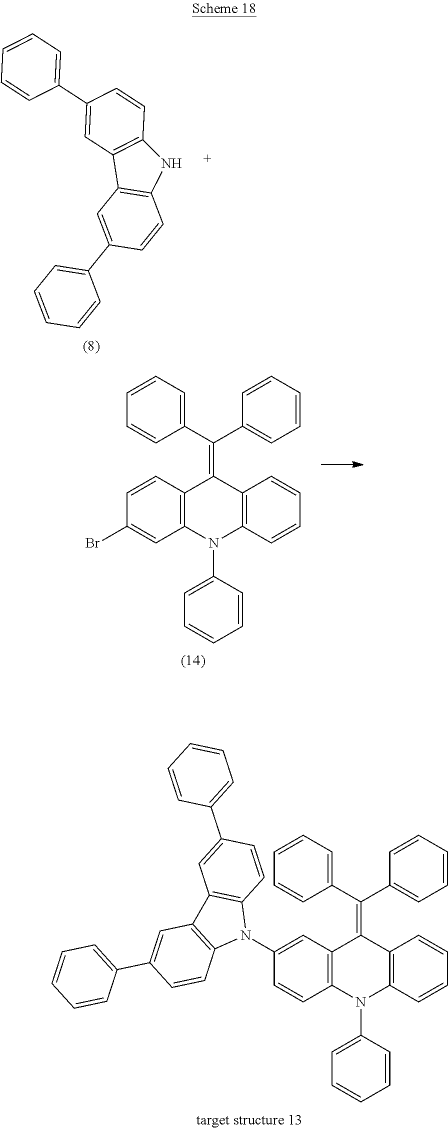 [SMILES: c1ccc(-c2ccc3[nH]c4ccc(-c5ccccc5)cc4c3c2)cc1 ; Brc1ccc2c(c1)N(c1ccccc1)c1ccccc1C2=C(c1ccccc1)c1ccccc1 ; c1ccc(C(=C2c3ccccc3N(c3ccccc3)c3ccc(-n4c5ccc(-c6ccccc6)cc5c5cc(-c6ccccc6)ccc54)cc32)c2ccccc2)cc1]

The method of preparing the tetrastyrene-based compound of the target structure 13 provided in Example 13 is as follows:

Compound (8) (4.0 mmol, 1.28 g), Compound (14) (5.0 mmol, 2.50 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 2.13 g of white powdery target structure 13, with a yield of 72%. Matrix-assisted laser desorption ionization time-of-flight mass spectrometer (MALDI-TOF): calculated value m/z, 738.93; measured value m/z, 738.69. Elemental analysis (EA): calculated value: carbon C, 91.03; hydrogen H, 5.18; nitrogen N, 3.79; measured value: C, 91.48; H, 5.21; N, 3.31.

### EXAMPLE 14

The synthesis reaction of the tetrastyrene-based compound of the target structure 14 is shown in the Scheme 19:

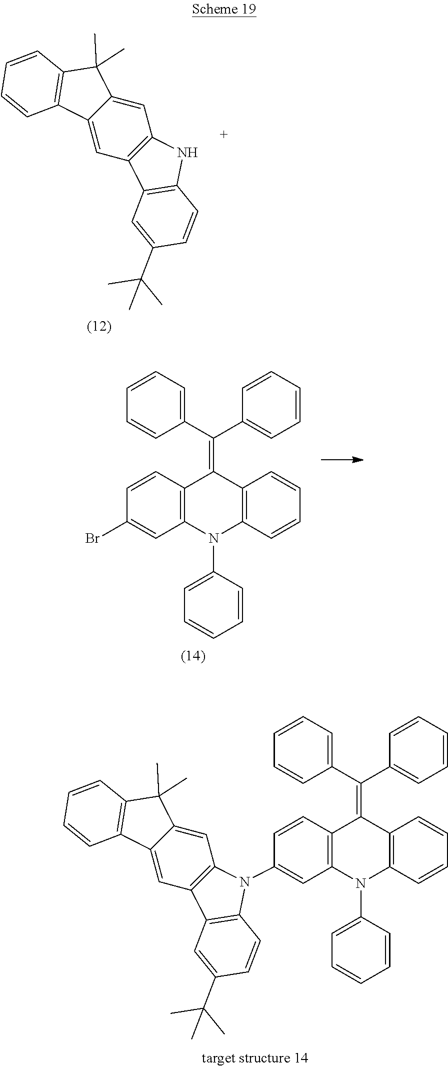 [SMILES: CC(C)(C)c1ccc2[nH]c3cc4c(cc3c2c1)-c1ccccc1C4(C)C ; Brc1ccc2c(c1)N(c1ccccc1)c1ccccc1C2=C(c1ccccc1)c1ccccc1 ; CC(C)(C)c1ccc2c(c1)c1cc3c(cc1n2-c1ccc2c(c1)N(c1ccccc1)c1ccccc1C2=C(c1ccccc1)c1ccccc1)C(C)(C)c1ccccc1-3]

The method of preparing the tetrastyrene-based compound of the target structure 14 provided in Example 14 is as follows:

Compound (12) (4.0 mmol, 1.30 g), Compound (14) (5.0 mmol, 2.50 g), t-BuONa (8 mmol, 0.76 g), Pd₂(dba)₃ (0.09 mmol, 81 mg), P(t-Bu)₃/HBF₄ (0.92 mmol, 0.24 g), and 50 mL anhydrous and deoxygenated toluene were added into a 100 mL two-necked flask, reacted at 98° C. overnight, and cooled to room temperature. Then, the reaction solution was concentrated, followed by separated and purified by column chromatography to obtain 1.88 g of white powdery target structure 14, with a yield of 62%. Matrix-assisted laser desorption ionization time-of-flight mass spectrometer (MALDI-TOF): calculated value m/z, 759.01; measured value m/z, 759.25. Elemental analysis method (EA): calculated value: carbon C, 90.20; hydrogen H, 6.11; nitrogen N, 3.69; measured value: C, 90.37; H, 6.35; N, 3.28.

### Test and Experimental Verification

After test and experimental verification, energy levels of the above-mentioned tetrastyrene-based compounds with target structures 1-14 are shown in Table 1 below:

TABLE 1

| tetrastyrene-based | Eg | HOMO | T1 |
|---|---|---|---|
| compound | (eV) | (eV) | (eV) |
| target structure 1 | 3.13 | −5.35 | 2.67 |
| target structure 2 | 3.23 | −5.34 | 2.71 |
| target structure 3 | 3.03 | −5.58 | 2.59 |
| target structure 4 | 3.29 | −5.62 | 2.73 |
| target structure 5 | 3.39 | −5.56 | 2.81 |
| target structure 6 | 3.1 | −5.37 | 2.65 |
| target structure 7 | 3.23 | −5.41 | 2.69 |
| target structure 8 | 3.38 | −5.63 | 2.74 |
| target structure 9 | 3.1 | −5.67 | 2.59 |
| target structure 10 | 3.42 | −5.65 | 2.87 |
| target structure 11 | 3.35 | −5.21 | 2.78 |
| target structure 12 | 3.37 | −5.25 | 2.81 |
| target structure 13 | 3.32 | −5.35 | 2.79 |
| target structure 14 | 3.35 | −5.37 | 2.78 |

An electronic device provided in an embodiment of the present application is manufactured according to a method known in the art. Taking an electroluminescent device as the electronic device as an example, the device structure may specifically include an ITO layer, a HAT-CN layer (for example, having a thickness of 5 nm), and an organic material layer of a tetrastyrene-based compound with any of the above target structures (for example, having a thickness of 30 nm), a Firpic:B3PyPB layer (12%, 10 nm), a TPBi layer (for example, having a thickness of 40 nm), a LiF layer (for example, having a thickness of 2 nm), and an aluminum Al layer (for example, having a thickness of 100nm). After test and experimental verification, for each of the above-mentioned tetrastyrene-based compounds of the target structures 1-14, the performance data of the electroluminescent devices whose organic material layers contain the tetrastyrene-based compounds of the target structures, respectively, is shown in Table 2 below:

TABLE 2

| | | Highest | Maximum external |
|---|---|---|---|
| organic material | Voltage | efficiency | quantum efficiency |
| layer | (v) | (cd/A) | (%) |
| target structure 1 | 3.4 | 26.3 | 12.1 |
| target structure 2 | 3.5 | 23.6 | 12.2 |
| target structure 3 | 3.8 | 21.5 | 11.3 |
| target structure 4 | 3.8 | 25.7 | 11.9 |
| target structure 5 | 3.9 | 19.5 | 11.2 |
| target structure 6 | 3.4 | 27.9 | 12.9 |
| target structure 7 | 3.5 | 33.2 | 13.5 |
| target structure 8 | 3.7 | 28.1 | 12.9 |
| target structure 9 | 3.9 | 19.7 | 9.6 |
| target structure 10 | 4.1 | 13.5 | 8.7 |
| target structure 11 | 3.2 | 31.5 | 12.8 |
| target structure 12 | 3.3 | 35.7 | 13.5 |
| target structure 13 | 3.1 | 31.4 | 13.2 |
| target structure 14 | 3.4 | 36.2 | 14.2 |

Embodiments of the present application provide a tetrastyrene-based compound, an application thereof, and an electronic device using the same, wherein the tetrastyrene-based compound includes an aromatic amine and a rigid tetrastyrene structure, the aromatic amine can effectively improve the hole injection and transport performance, thereby improving the balance of electrons and holes of the organic light-emitting diode, achieving a lower voltage and higher efficiency; and the rigid tetrastyrene structure is conducive to the formation of evaporation materials of melting type, thereby being conducive to the stability of mass production evaporation. Such materials can achieve high-efficiency preparation of electroluminescent devices, and can be used in the manufacture of display devices

While the invention has been described by way of example and in terms of the preferred embodiments, it is to be understood that the invention is not limited to the disclosed embodiments. To the contrary, it is intended to cover various modifications and similar arrangements. Therefore, the scope of the appended claims should be accorded the broadest interpretation so as to encompass all such modifications and similar arrangements.

What is claimed is:

1. A tetrastyrene-based compound, having a general structural formula as shown in the following Formula 1: 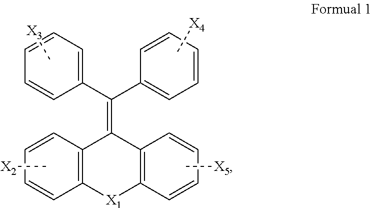
wherein, in Formula 1, X1 is selected from, 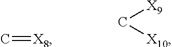
O, N-X₁₁, or S;
X2, X3, X4, and X5 are each independently selected from an arylamine with a general structural formula as shown in Formula 2, a hydrogen, an alkyl group having 1 to 22 carbon atoms, an alkoxy group having 1 to 22 carbon atoms, or a heteroalkyl group having 1 to 22 carbon atoms, a mono- or multi-substituted or unsubstituted aryl group, or a substituted or unsubstituted heteroaryl group, wherein a heteroatom of the heteroalkyl group is O, N, F, S, or Si, a heteroatom of the heteroaryl group is Si, Ge, N, P, O, S, or Se, and at least one of X2, X3, X4, and X5 is an arylamine with the general structural formula as shown in Formula 2: 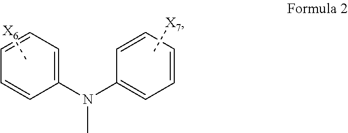
X6, X7, X8, X9, X10, and X11 are each independently selected from hydrogen, an alkyl group, an alkoxy group having 1 to 22 carbon atoms, or a heteroalkyl group having 1 to 22 carbon atoms, a mono- or multi-substituted or unsubstituted aryl group, or a substituted or unsubstituted heteroaryl group, wherein a heteroatom of the heteroalkyl group is O, N, F, S, or Si, and a heteroatom of the heteroaryl group is Si, Ge, N, P, O, S, or Se, or at least two of X2, X3, X4, X5, X6, X7, X8, X9, X10, and X11 are connected to each other to form a monocyclic ring or fused ring of an aromatic ring or a heterocyclic ring when the at least two of X2, X3, X4, X5, X6, X7, X8, X9, X10, and X11 are adjacent aryl groups or heteroaryl groups; and
when expressed as “substituted or unsubstituted”, optional substituents of each of the aryl group and the heteroaryl group are each independently selected from H, halogen, —OH, —SH, —CN, —NO₂, and an alkylthio group having 1 to 15 carbon atoms, an alkyl group having 1 to 40 carbon atoms, or a substituted alkyl group having 1 to 40 carbon atoms.

2. The tetrastyrene-based compound according to claim 1, wherein a general structural formula of X1 is 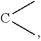
and a general structural formula of the tetrastyrene-based compound is shown in the following Formula 3: 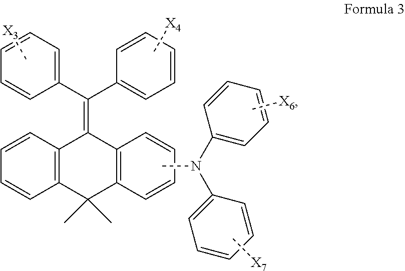
wherein, in Formula 3, X3, X4, X6, and X7 are each independently selected from a mono- or multi-substituted, or unsubstituted aryl group, or a substituted or unsubstituted heteroaryl group, or at least two of X3, X4, X6, and X7 are connected to each other to form a monocyclic ring or fused ring of an aromatic ring or a heterocyclic ring when the at least two of X3, X4, X6, and X7 are adjacent aryl groups or heteroaryl groups.

3. The tetrastyrene-based compound according to claim 2, wherein a structure of the tetrastyrene-based compound is represented by any one of the following general Formulas 301-320:

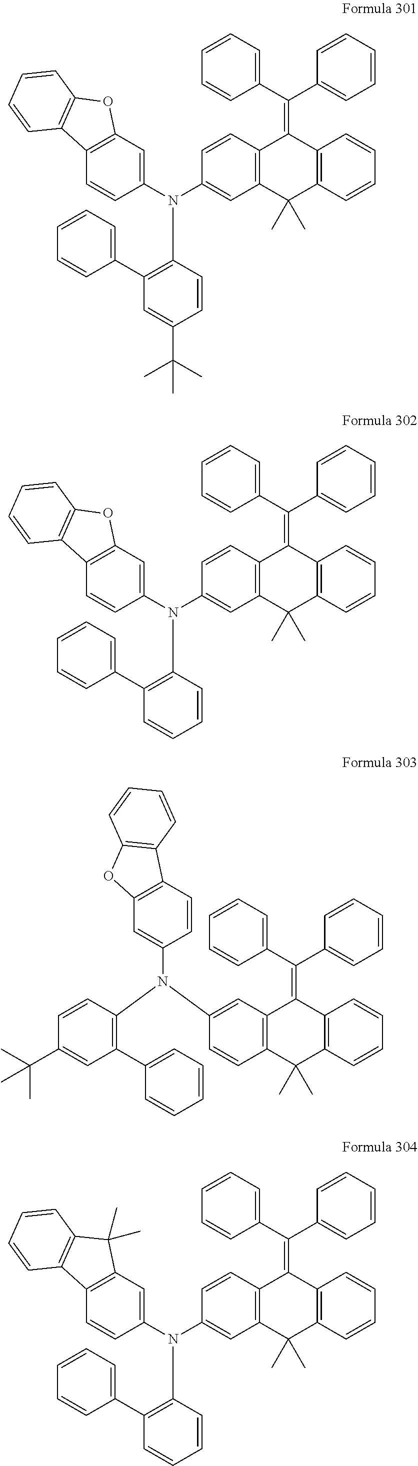

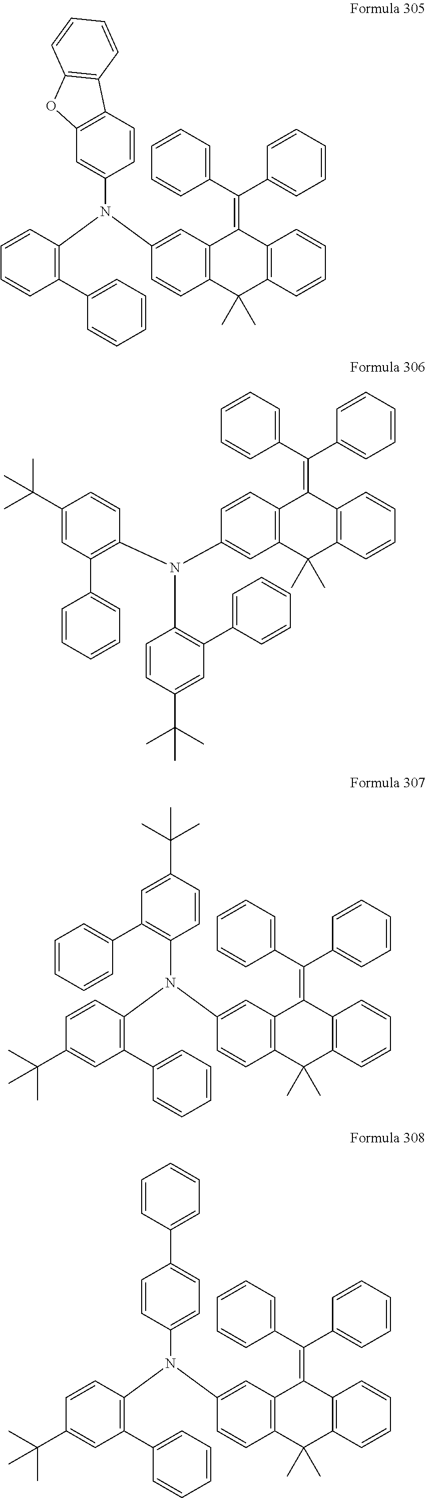

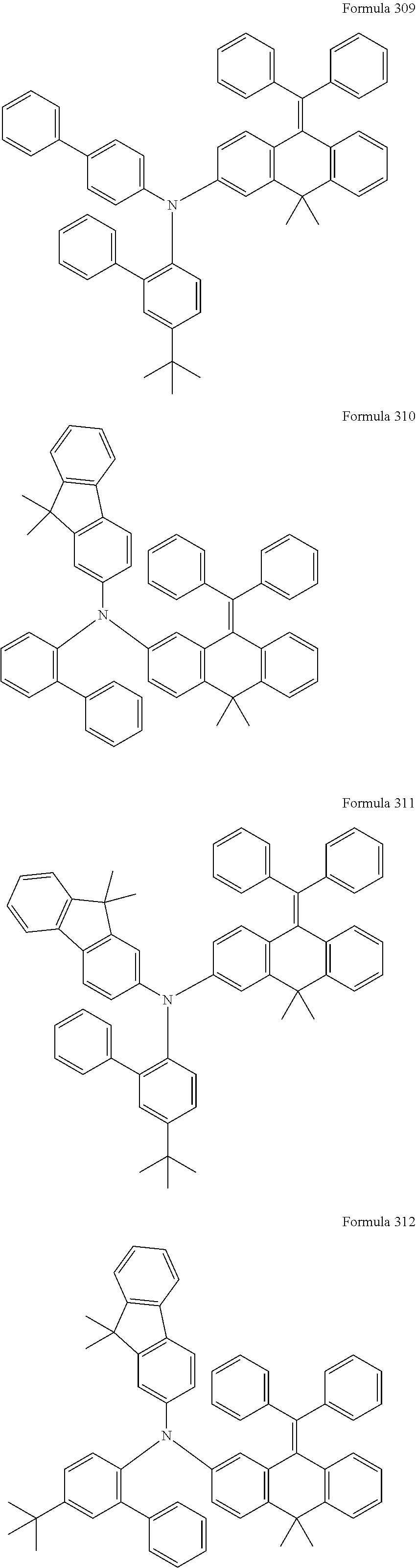

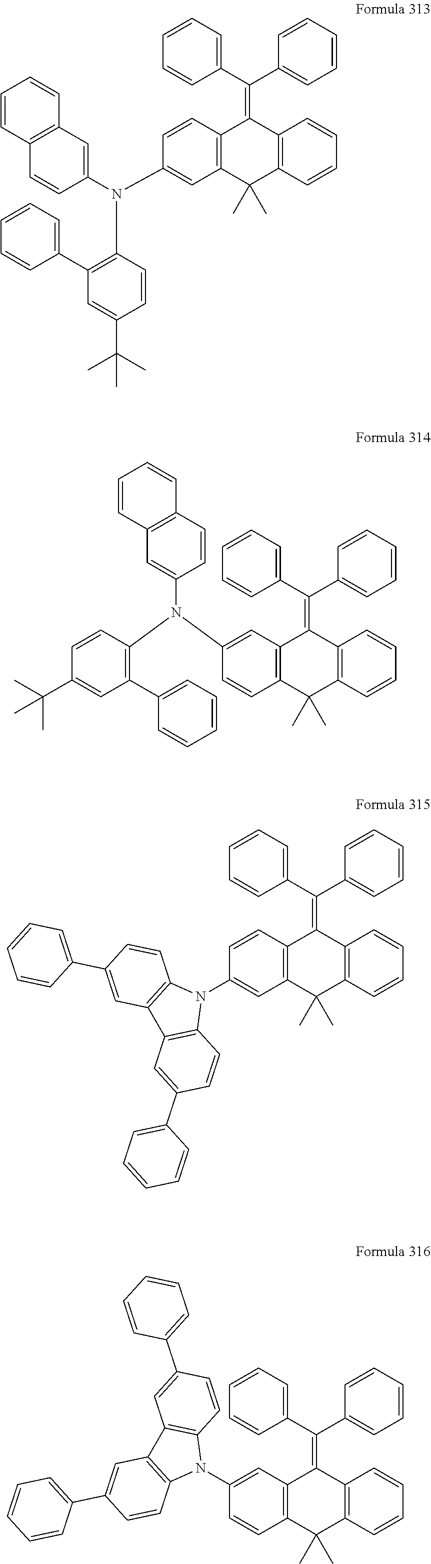

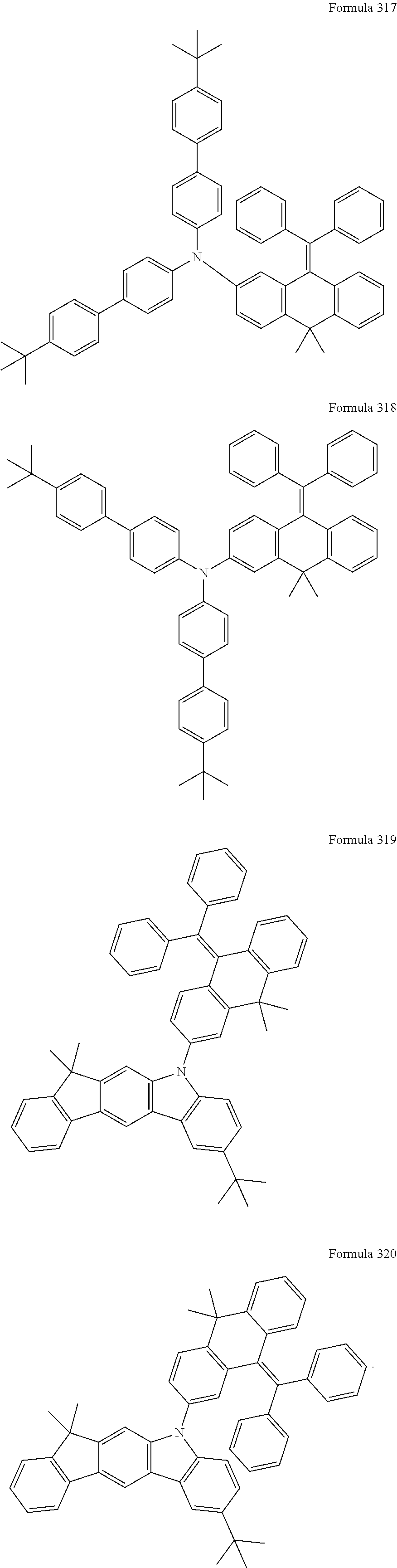

4. The tetrastyrene-based compound according to claim 1, wherein a general structural formula of X1 is O, and a general structural formula of the tetrastyrene-based compound is shown in the following Formula 4: 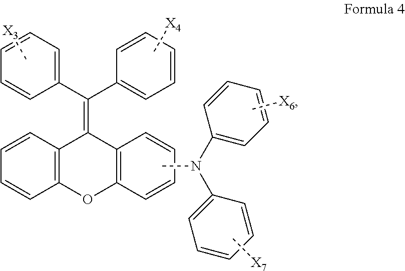
wherein, in Formula 4, X3, X4, X6, and X7 are each independently selected from a mono- or multi-substituted, or unsubstituted aryl group, or a substituted or unsubstituted heteroaryl group, or at least two of X3, X4, X6, and X7 are connected to each other to form a monocyclic ring or fused ring of an aromatic ring or a heterocyclic ring when the at least two of X3, X4, X6, and X7 are adjacent aryl groups or heteroaryl groups.

5. The tetrastyrene-based compound according to claim 4, wherein a structure of the tetrastyrene-based compound is represented by any one of the following general Formulas 401-420:

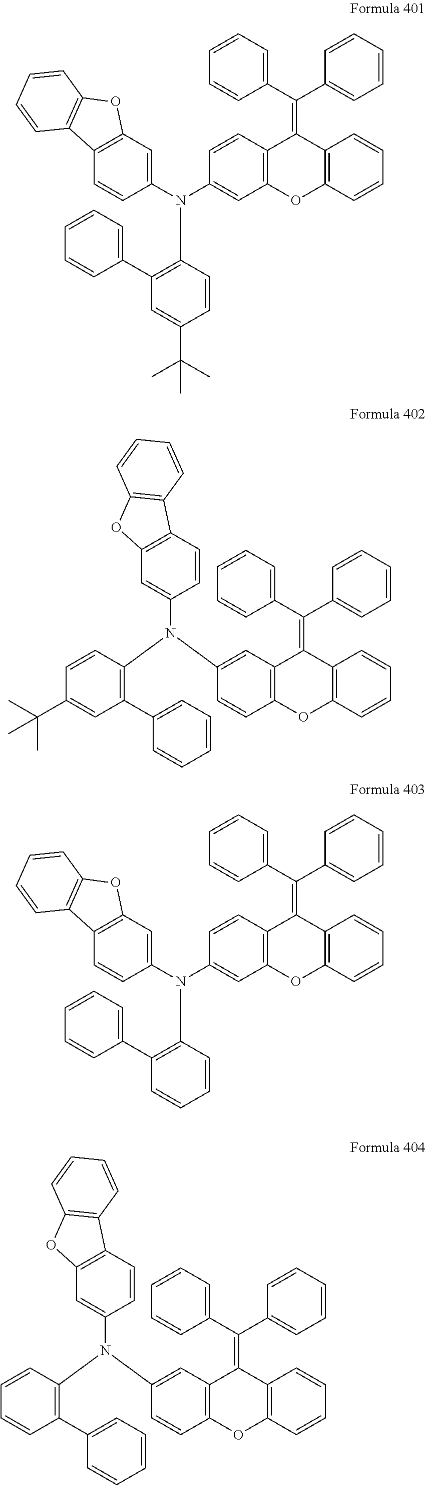

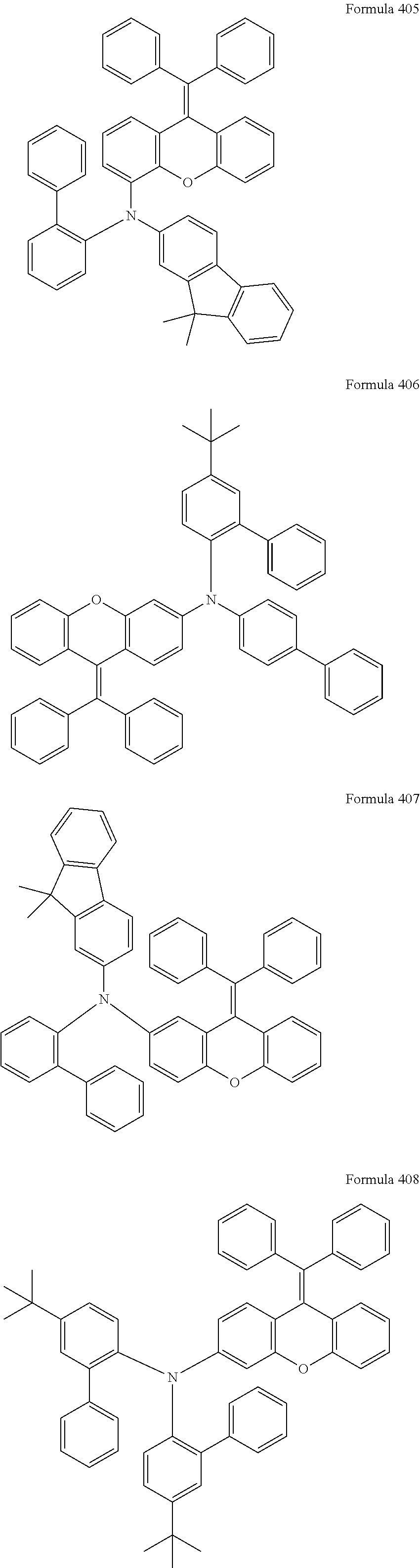

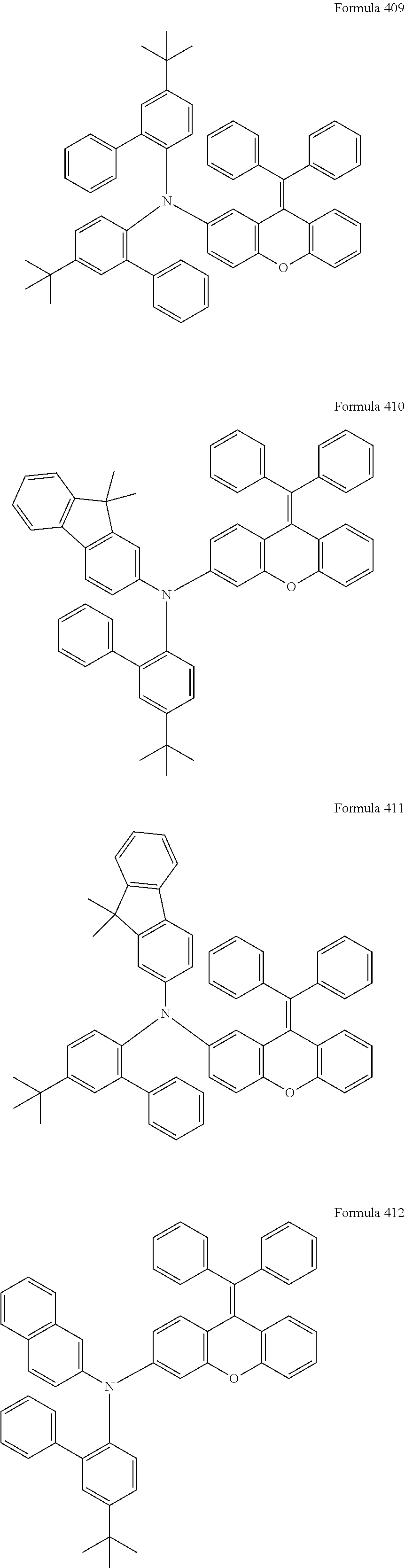

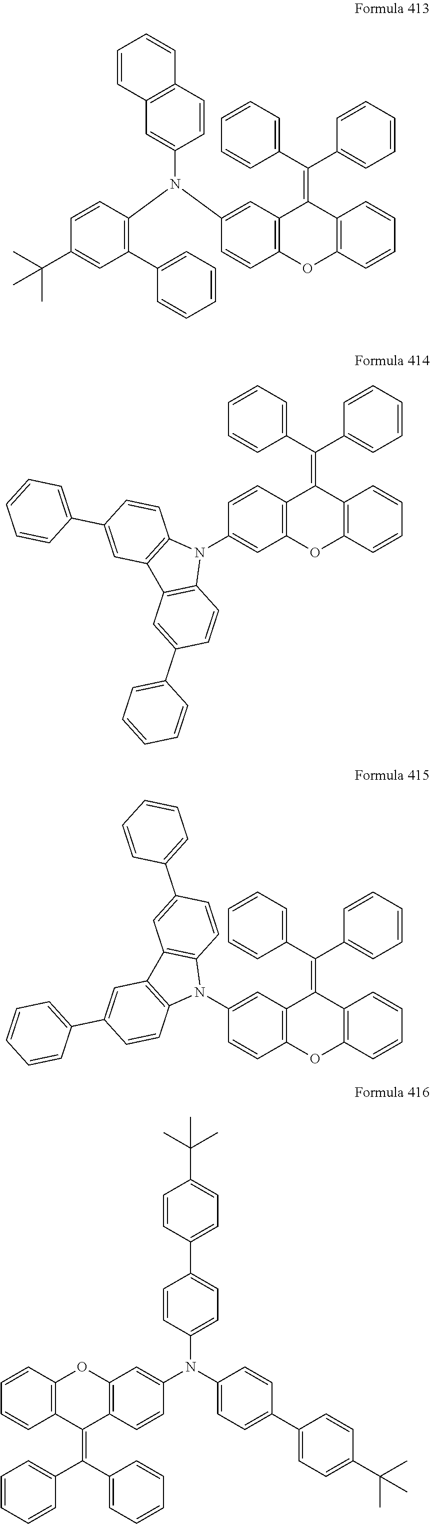

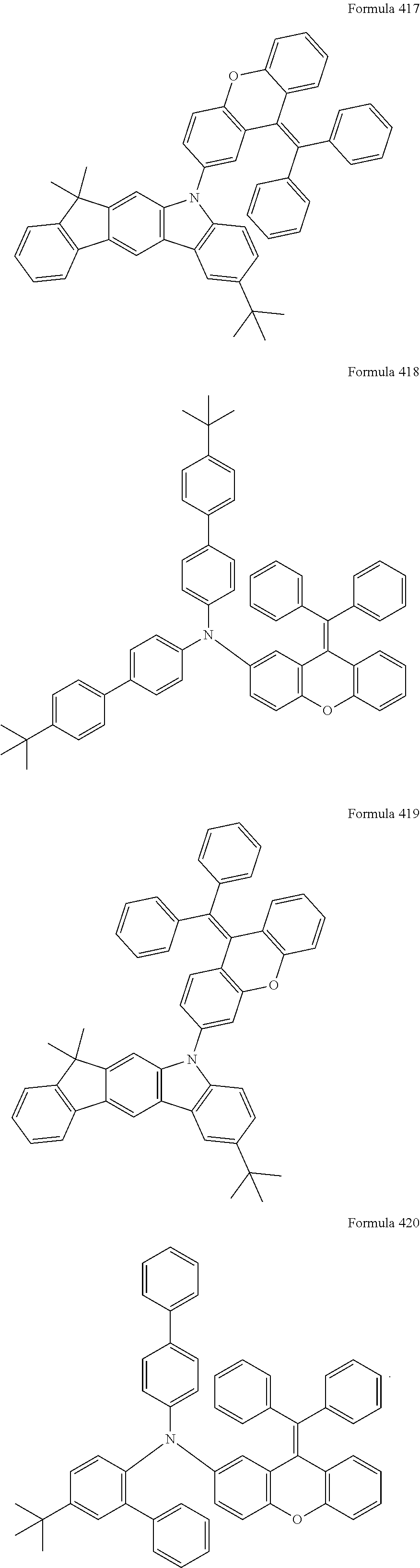

6. The tetrastyrene-based compound according to claim 1, wherein a general structural formula of X1 is N-ph, and a general structural formula of the tetrastyrene-based compound is shown in Formula 5 below: 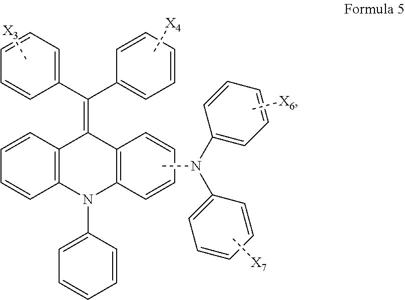
wherein, in Formula 5, X3, X4, X6, and X7 are each independently selected from a mono- or multi-substituted, or unsubstituted aryl group, or a substituted or unsubstituted heteroaryl group, or at least two of X3, X4, X6, and X7 are connected to each other to form a monocyclic ring or fused ring of an aromatic ring or a heterocyclic ring when the at least two of X3, X4, X6, and X7 are adjacent aryl groups or heteroaryl groups.

7. The tetrastyrene-based compound according to claim 6, wherein a structure of the tetrastyrene-based compound is represented by any one of the following general Formulas 501-520:

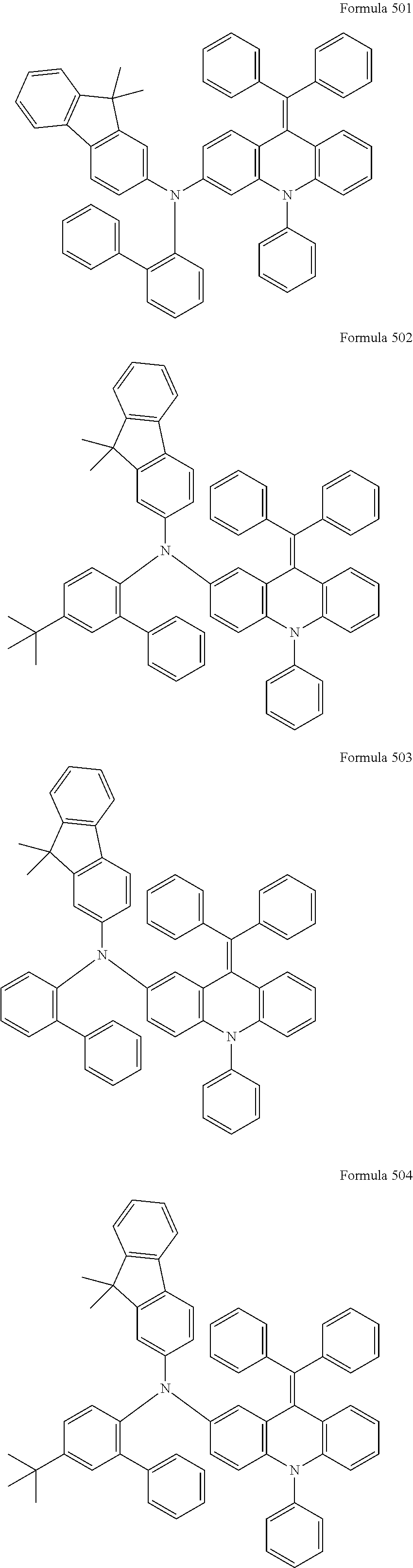

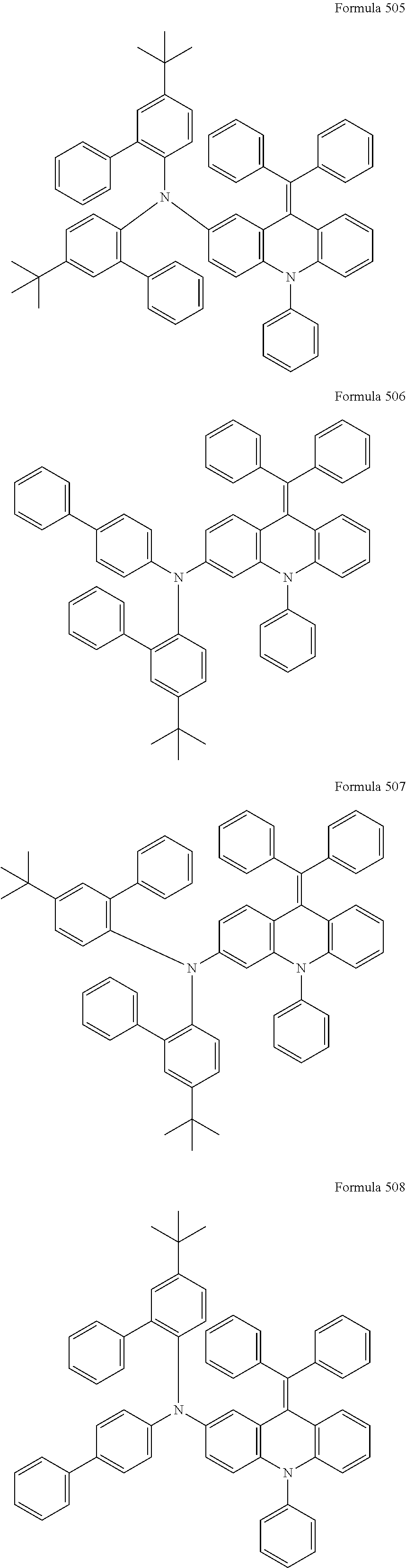

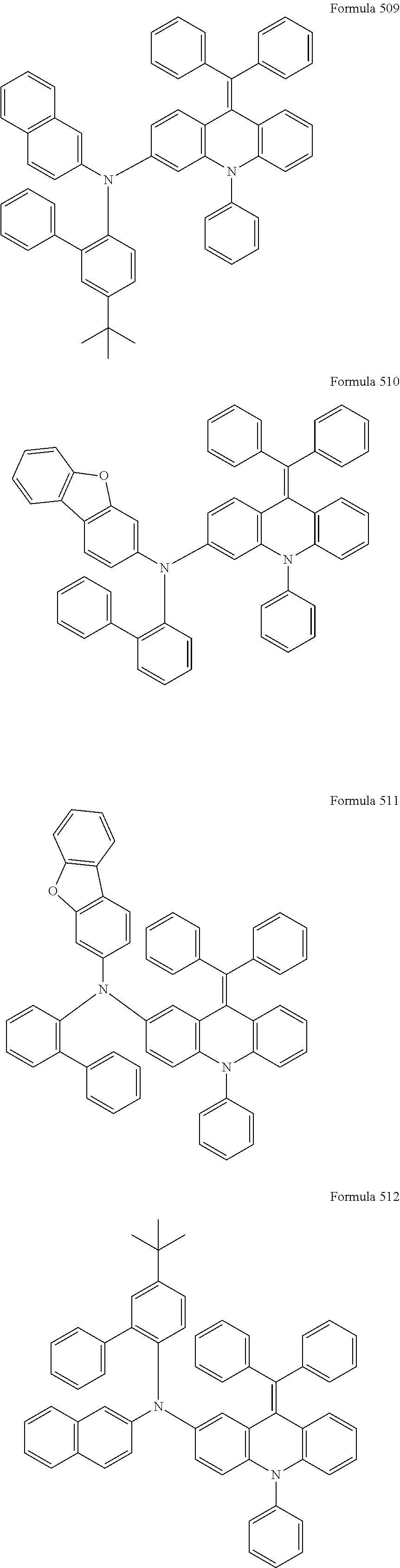

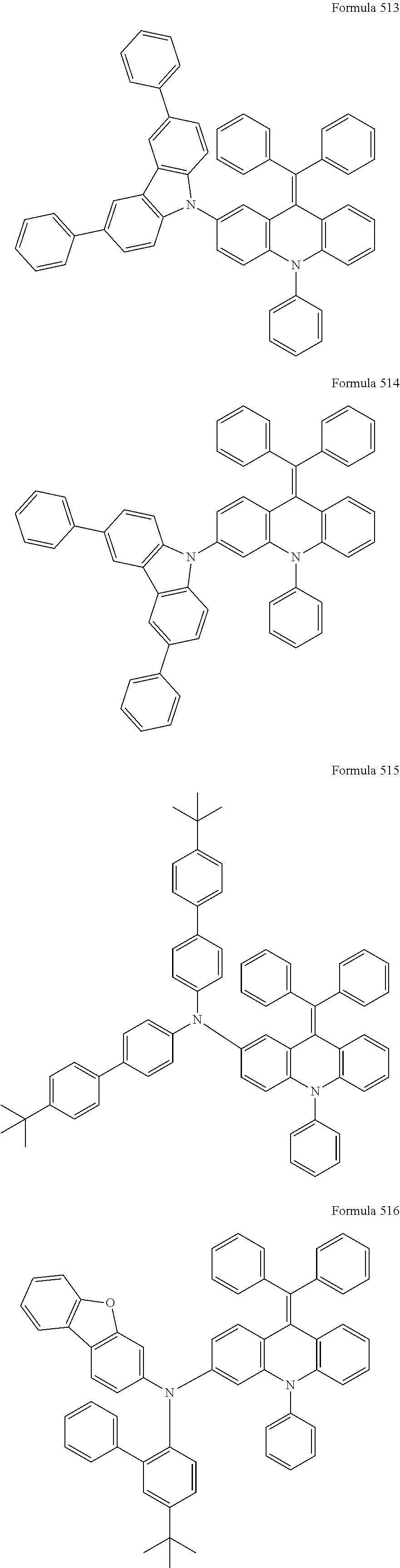

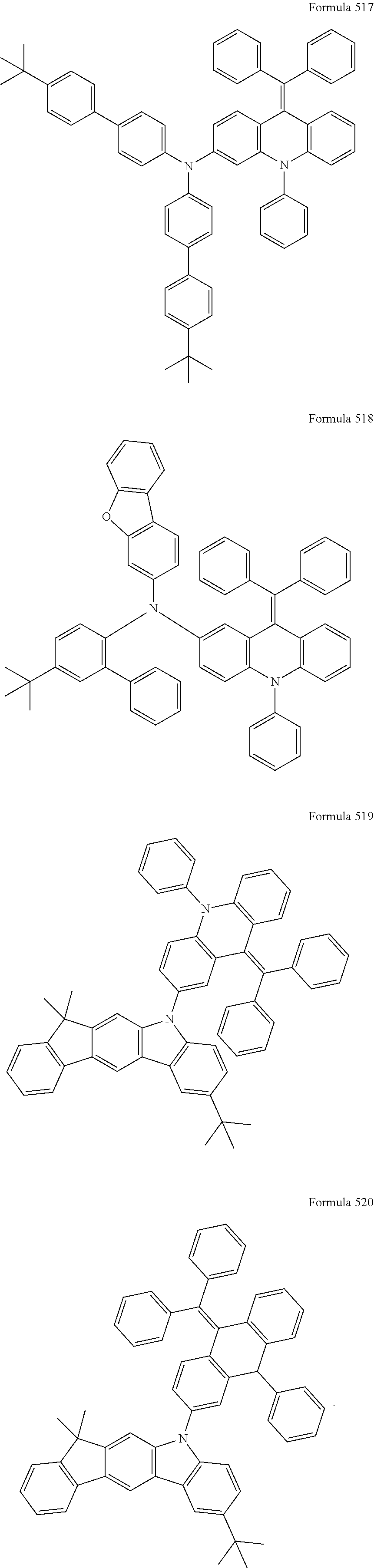

8. An application of the tetrastyrene-based compound according to claim 1 as an electroluminescent organic material in an electronic device.

9. An electronic device, comprising a substrate, an anode, a cathode, and one or more organic material layers interposed between the anode and the cathode, wherein at least one of the one or more organic material layers containing the tetrastyrene-based compound according to claim 1.

10. The electronic device according to claim 9, wherein the organic material layer comprises a hole injection layer, a hole transport layer, an electron injection layer, an electron transport layer, and a light-emitting layer.
